(19) United States
(12) Patent Application Publication
(10) Pub. No.: US 2026/0009727 A1
(43) Pub. Date: Jan. 8, 2026

(54) DETERMINING HYDROCARBON EFFLUENT COMBUSTION EFFICIENCY

(71) Applicant: SCHLUMBERGER TECHNOLOGY CORPORATION, Sugar Land, TX (US)

(72) Inventor: Sebastien CATHELINE, Clamart (FR)

(21) Appl. No.: 19/137,308

(22) PCT Filed: Dec. 18, 2023
(86) PCT No.: PCT/US2023/084508

§ 371 Date: Jun. 10, 2025

Related U.S. Application Data
(60) Provisional application No. 63/476,957, filed on Dec. 23, 2022.

(51) Int. Cl. G01N 21/39; E21B 41/00; G01N 33/00

(52) U.S. Cl. CPC G01N 21/39; E21B 41/0071; G01N 33/004; G01N 33/0047

(57) ABSTRACT

A gas monitoring system for determining a property of a gas plume produced by burning of a hydrocarbon effluent via a burning device. The gas monitoring system can include a laser emission system operable to emit a laser beam along a plurality of paths passing through the gas plume. The system also includes a detection system operable to facilitate determining intensity data indicative of intensities of the laser beam that has been backscattered by a surface after passing through the gas plume, and a processing system with computer program code. The computer program configured to control laser emission system, output concentration path length; discretize the concentration path length data in the form of a concentration path length map, find a plume region of the concentration path length map; and determine mean concentration path length of the predetermined gas.

### CROSS-REFERENCE TO RELATED APPLICATIONS

The present application claims priority benefit of U.S. Provisional Application No. 63/476,957, filed Dec. 23, 2022, the entirety of which is incorporated by reference herein and should be considered part of this specification.

### BACKGROUND OF THE DISCLOSURE

The global oil and gas industry is trending toward improved environmental safety and compliance throughout various phases of a well lifecycle. During exploration and appraisal of new oil and gas fields, wells are drilled and tested to assess the commercial viability of these fields. Dynamic well testing can produce a large amount of hydrocarbons to a wellsite surface. However, excess hydrocarbons cannot be stored, and hydrocarbon disposal is difficult due to the lack of transport infrastructure at well sites. Such problems are even more relevant in offshore operations. Thus, the most economical viable option is often to dispose of the excess hydrocarbons by burning the hydrocarbons, compromising between optimal environmental and financial constraints.

Burning hydrocarbons produces pollutant gases, such as carbon monoxide (CO), carbon dioxide (CO₂), nitric oxide (NO), nitrogen dioxide (NO₂), nitrogen trioxide (NO₃), and/or sulfur dioxide (SO₂), as well as residual unburned hydrocarbon, such as methane (CH₄), acetylene (C₂H₂), ethylene (C₂H₄), and propylene (C₃H₆). Releasing these pollutant gases into the atmosphere exacerbates the greenhouse effect, and environmental protection agencies scrutinize such releases and often require periodic reporting of the quantities of these pollutant gases that have been released into the atmosphere.

### BRIEF DESCRIPTION OF THE DRAWINGS

The present disclosure is understood from the following detailed description when read with the accompanying figures. It is emphasized that, in accordance with the standard practice in the industry, various features are not drawn to scale. In fact, the dimensions of the various features may be arbitrarily increased or reduced for clarity of discussion.

FIG. 1 is a schematic view of at least a portion of an example environment in which one or more aspects of the present disclosure may be implemented.

FIG. 2 is a schematic view of at least a portion of an example implementation of apparatus according to one or more aspects of the present disclosure.

FIG. 3 is a schematic view of at least a portion of an example implementation of apparatus according to one or more aspects of the present disclosure.

FIG. 4 is a schematic view of at least a portion of an example implementation of apparatus according to one or more aspects of the present disclosure.

FIG. 5 is a graph according to one or more aspects of the present disclosure.

FIG. 6 is a schematic view of a portion of an example implementation of the apparatus shown in FIG. 4 according to one or more aspects of the present disclosure.

FIG. 7 is a schematic view of a portion of another example implementation of the apparatus shown in FIG. 4 according to one or more aspects of the present disclosure.

FIG. 8 is a schematic view of at least a portion of an example implementation of apparatus according to one or more aspects of the present disclosure.

FIG. 9 is a schematic view of at least a portion of an example implementation of apparatus according to one or more aspects of the present disclosure.

FIG. 10 is a schematic view of at least a portion of an example implementation of apparatus according to one or more aspects of the present disclosure.

FIG. 11 is a flow-chart diagram of at least a portion of a method according to one or more aspects of the present disclosure.

FIG. 12 is a schematic view of at least a portion of a method according to one or more aspects of the present disclosure.

FIG. 13 is a flow-chart diagram of at least a portion of a method according to one or more aspects of the present disclosure.

FIG. 14 is a schematic view of at least a portion of an example implementation of apparatus according to one or more aspects of the present disclosure.

FIG. 15 is a graph according to one or more aspects of the present disclosure.

FIG. 16 is a schematic view of at least a portion of an example implementation of apparatus according to one or more aspects of the present disclosure.

FIG. 17 is a schematic view of at least a portion of an example implementation of apparatus according to one or more aspects of the present disclosure.

FIG. 18 is a graph according to one or more aspects of the present disclosure.

FIG. 19 is a graph according to one or more aspects of the present disclosure.

FIG. 20 is a graph according to one or more aspects of the present disclosure.

FIG. 21 is a schematic view of at least a portion of an example implementation of apparatus according to one or more aspects of the present disclosure.

### DETAILED DESCRIPTION

It is to be understood that the following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for simplicity and clarity, and does not in itself dictate a relationship between the various embodiments and/or configurations discussed. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact.

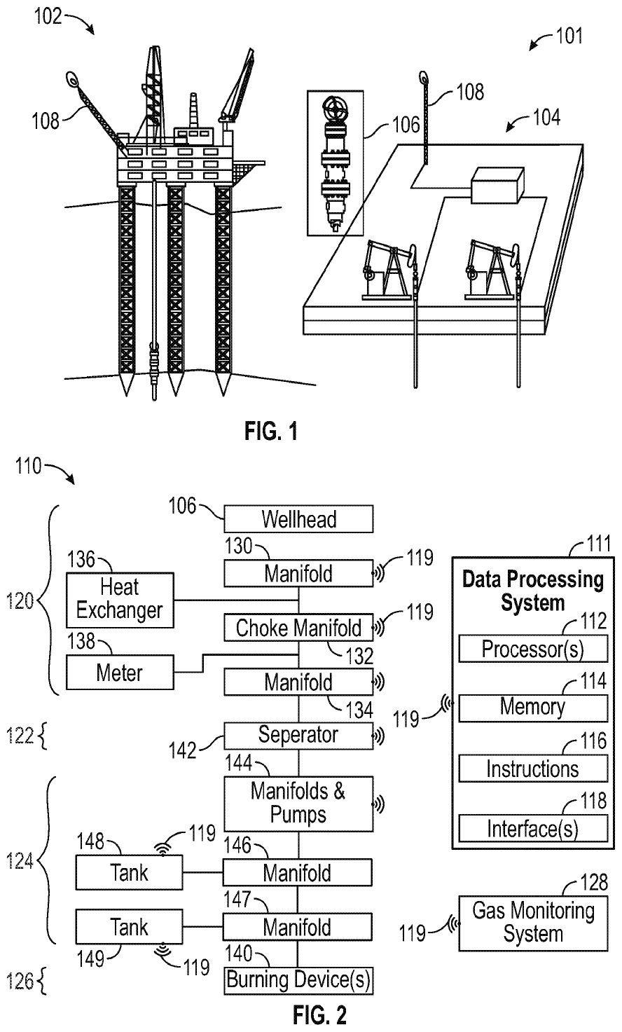

FIG. 1 depicts schematic views of an example marine environment 102 (e.g., an offshore oil and gas well drilling and production rig) and an example land environment 104 (e.g., a land-based oil pumping station) related to one or more aspects of the present disclosure. The marine environment 102 and land environment 104 represent example environments 101 in which one or more aspects of the present disclosure described below may be implemented. Although not shown, the example environments 101 may also include a land-based oil and gas well drilling rig. Each of the environments 101 may include one or more surface well terminations, known as wellheads 106, each installed over and sealing a corresponding wellbore. For example, a wellhead 106 may be at a land surface or a subsea surface (e.g., an ocean bottom). Each wellhead 106 may include a system of spools, valves, and assorted adapters that, for example, can provide for pressure control of a production well. Each wellhead 106 may comprise various types of wellhead equipment, such as casing and tubing heads, a production tree, and a blowout preventer, among other examples. Conduits from multiple wellheads 106 may be joined at one or more manifolds such that well fluid from multiple wells can flow in a common conduit.

At various times, a well may be tested. Well testing can include one or more of a variety of well testing operations. In various instances, well fluid (e.g., oil and/or gas) can flow from one or more wells to a wellsite surface where the well fluid is subjected to one or more well testing operations which generate scrap (e.g., waste fluid) to be handled according to governmental regulations and/or location/operator-specific circumstances. For example, waste fluid may be loaded into a tanker for transport to a facility that can dispose of the waste fluid. Waste fluid may also or instead be disposed of via burning (i.e., combustion), which can include burning waste oil and/or flaring waste gas. Burning can also or instead be part of the well testing operations, such as for analyzing well fluid, which may provide data indicative of composition and/or other characteristics of the well fluid. For example, well testing operations can be performed during one or more phases, such as during exploration and appraisal, where production of hydrocarbons are tested using a temporary production facility that can facilitate well fluid sampling, flow rate analysis, and pressure information, such as to help characterize a reservoir. Various decisions can be based on well testing, such as decisions related to production methods and/or well productivity improvements. For example, hydrocarbons produced during well testing may be disposed of via burning operations, which can include on-site and/or off-site burning. Burning well fluid as part of well testing and/or waste fluid disposal operations may be performed by one or more burning devices 108, such as oil burners and/or gas flares.

Well testing may be performed, for example, using equipment shown in the marine environment 102 and/or the land environment 104. As an example, an environment may be under exploration, development, and/or appraisal, where such an environment includes at least one well that can produce a well fluid (e.g., via natural pressure, fracturing, artificial lift, pumping, flooding, etc.). In such an environment, various types of equipment may be on-site, which may be operatively coupled to the well testing equipment.

FIG. 2 shows an example implementation of a well fluid processing system 110 related to one or more aspects of the present disclosure and represents an example environment in which one or more aspects of the present disclosure described below may be implemented. The well fluid processing system 110 may be or form at least a portion of a well fluid testing system or otherwise be utilized for testing and disposing of fluid received from a well.

The well fluid processing system 110 may be fluidly connected with and receive well fluid via a wellhead 106. The well fluid processing system 110 may also or instead be fluidly connected with and receive well fluid via other fluid conduits for transporting well fluid discharged from a well. The well fluid processing system 110 may comprise a data processing (i.e., computing) system 111, which may include one or more processors 112, memory 114 accessible to at least one of the one or more processors 112, instructions (i.e., a computer program code) 116 that can be stored in the memory 114 and executed by at least one of the one or more processors 112, and one or more communication interfaces 118. The well fluid processing system 110 may comprise various wired and/or wireless communication means 119 operable to transmit and/or receive information (e.g., sensor data, control commands, etc.), for example, to and/or from the data processing system 111. The data processing system 111 may be or comprise a controller that can issue control instructions to one or more pieces of equipment in an environment, such as the marine environment 102 and/or the land environment 104 depicted in FIG. 1. The data processing system 111 may be local, remote, or distributed (e.g., partially local and partially remote).

The well fluid processing system 110 may comprise various segments that may be categorized operationally, such as a well control segment 120, a separation segment 122, a fluid management segment 124, and a burning segment 126. The well control segment 120 may comprise an assembly of various components, such as a manifold 130 connected to the wellhead 106, a choke manifold 132, a heat exchanger 136 connected between the manifold 130 and the choke manifold 132, a manifold 134, and a meter 138 connected between the choke manifold 132 and the manifold 134. The separation segment 122 may comprise a separator 142 connected to the manifold 134. The fluid management segment 124 may comprise an assembly of various components, such as manifolds and pumps 144 connected to the separator 142, a tank 148, a manifold 146 connected between the tank 148 and the manifolds and pumps 144, a tank 149, and a manifold 147 connected between the tank 149 and the manifold 146. The burning segment 126 may comprise one or more burning devices 140 (i.e., combustion devices) connected to the manifold 147.

The well fluid processing system 110 may comprise or operate in conjunction with a gas monitoring system 128 operable to monitor various properties of a gas plume produced by the burning well fluids (e.g., oil and/or gas) during burning operations performed by the one or more burning devices 140 (e.g., oil burners, gas flares, etc.). For example, the gas monitoring system 128 may be operable to monitor concentrations and/or rates at which various individual component gases within or forming the gas plume are produced (i.e., emitted) by the burning of the well fluids via the one or more burning devices 140. The gas monitoring system 128 may also or instead be operable to monitor efficiency of burning (i.e., combustion efficiency) of the well fluids by the one or more burning devices 140. At least a portion of the gas monitoring system 128 may be located and/or operate in association with the one or more burning devices 140, such as may permit the gas monitoring system 128 to monitor the various properties of the gas plume. The data processing system 111 may be or form a portion of the gas monitoring system 128 or otherwise operate in conjunction with the gas monitoring system 128. The data processing system 111 may be communicatively connected with the gas monitoring system 128 and may be operable to receive and process sensor data from the gas monitoring system 128.

The well fluid processing system 110 may comprise various features for performing well testing operations, including fewer features, more features, and/or alternative features than as shown in FIG. 2. For example, the well fluid processing system 110 may comprise one or more of a gas specific gravity meter, a water-cut meter, a gas-to-oil ratio sensor, a carbon dioxide sensor, a hydrogen sulfide sensor, or a shrinkage measurement device. Various features may be upstream and/or downstream of the separator segment 122 or the separator 142.

The flow of a well fluid containing hydrocarbons from a well via the wellhead 106 may be received by the well control segment 120 and then routed via one or more conduits to the separation segment 122. The heat exchanger 136 of the well control segment 120 may be implemented as a steam-heat exchanger. The meter 138 may be operable to measure flow of well fluid through the well control segment 120. The well fluid from the well may be a single phase or multiphase fluid (i.e., two or more of oil, water, and gas). The well control segment 120 may convey the well fluid received from one or more wells to the separator 142, which may comprise one or more features for facilitating separation of components of incoming well fluid (e.g., diffusers, mist extractors, vanes, baffles, precipitators, etc.). The separator 142 may be a horizontal separator or a vertical separator. The separator 142 may be a two-phase separator (e.g., for separating gases and/or liquids) or a three-phase separator (e.g., for separating gas, oil, and/or water). The separator 142 may be used to substantially separate multiphase fluid into its oil, gas, and water phases, wherein each phase emerging from the separator 142 may be referred to herein as a separated well fluid. Such separated well fluids may be routed away from the separator 142 to the fluid management segment 124. The separated well fluids may not be entirely homogenous. For example, separated gas exiting the separator 142 may include some residual amount of water or oil, separated water exiting the separator 142 may include some amount of oil or entrained gas, and separated oil leaving the separator 142 may include some amount of water or entrained gas.

The well fluid management segment 124 may include flow control equipment, such as various manifolds and pumps 144 for receiving well fluids from the separator 142 and conveying the well fluids to other destinations, including additional manifolds 146, 147 for routing the well fluid to and from fluid tanks 148, 149. Although the fluid management segment 124 is shown comprising two manifolds 146, 147 and two tanks 148, 149, the fluid management segment 124 may comprise a different number of manifolds 146, 147 and tanks 148, 149. For example, the fluid management segment 124 may comprise a single manifold and a single tank, or the fluid management segment 124 may comprise more than two manifolds and/or more than two tanks. The manifolds and pumps 144 may comprise a variety of manifolds and pumps, such as a gas manifold, an oil manifold, an oil transfer pump, a water manifold, and/or a water transfer pump. The manifolds and pumps 144 may be used to route well fluids received from the separator 142 to one or more of the fluid tanks 148, 149 via one or more of the additional manifolds 146, 147, and to route well fluids between the tanks 148, 149. The manifolds and pumps 144 may comprise features for routing well fluids received from the separator 142 directly to the one or more burning devices 140 for burning gas and oil (e.g., bypassing the tanks 148, 149) or for routing well fluids from one or more of the tanks 148, 149 to the one or more burning devices 140.

As noted above, components of the well fluid processing system 110 may vary between different applications and/or equipment within each functional group, or the well fluid processing system 110 may vary between different applications. For example, the heat exchanger 136 may be provided as part of the separation segment 122 instead of the well control segment 120.

The well fluid processing system 110 may form at least a portion of or operate in conjunction with a surface well testing system. The well fluid processing system 110 may be monitored and controlled remotely, such as via sensors and actuators installed in association with the segments 120, 122, 124, 126 and/or individual components of the well fluid processing system 110. For example, a dedicated monitoring system (e.g., sensors, communication systems, human-machine interfaces, etc.) may facilitate monitoring of one or more of the segments 120, 122, 124 126.

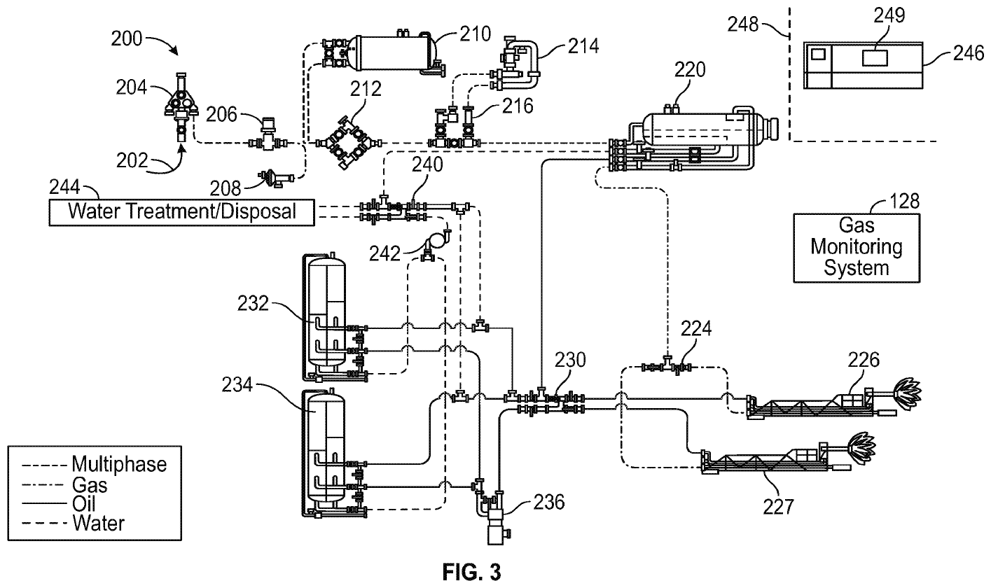

FIG. 3 shows an example implementation of a well fluid processing system 200 according to one or more aspects of the present disclosure. The system 200 may be utilized for testing a well fluid received from a well or the system 200 may be or form at least a portion of a well fluid testing system. The system 200 may be an example implementation of the well fluid processing system 110 shown in FIG. 2 or the system 200 may comprise one or more features of the well fluid processing system 110. The area in which the system 200 is installed may be classified as a hazardous area. In some implementations, the well test area may be classified as a Zone 1 hazardous area according to International Electrotechnical Commission (IEC) standard 60079-10-1:2015.

The system 200 may receive a multiphase well fluid (represented by arrow 202) from a well via a flowhead 204. The well fluid 202 may then be directed to a separator 220 through a surface safety valve 206, a steam-heat exchanger 210, a choke manifold 212, a flow meter 214, and an additional manifold 216. The system 200 may further comprise a chemical injection pump 208 for injecting chemicals into the multiphase well fluid flowing toward the separator 220. The separator 220 may be a three-phase separator operable to separate the multiphase well fluid 202 into gas, oil, and water components.

The separated gas may be directed downstream from the separator 220 through a gas manifold 224 to either of burning devices 226, 227 for burning (i.e., flaring). The gas manifold 224 may comprise valves that can be actuated to control gas flow from the gas manifold 224 to one or the other of the burning devices 226, 227 (e.g., oil burners, gas flares, etc.). Although the burning devices 226, 227 are shown adjacent each other for the sake of clarity, the burning devices 226, 227 may be positioned apart from each other, such as on opposite sides of a rig or other wellsite installation. The separated oil from the separator 220 may be directed downstream to an oil manifold 230 comprising valves that can be operated to permit oil flow to either of the tanks 232, 234 or to either of the burning devices 226, 227 for burning. The tanks 232, 234 may be or comprise vertical surge tanks, each having two fluid compartments, or the tanks 232, 234 may comprise other suitable forms. Each tank 232, 234 may be configured to simultaneously hold different well fluids, such as water in a first compartment and oil in a second compartment. An oil transfer pump 236 may be operated to pump oil through the system 200 downstream of the separator 220. The separated water from the separator 220 may be directed to a water manifold 240. Like the oil manifold 230, the water manifold 240 may comprise valves that can be opened or closed to permit water flow to either of the tanks 232, 234 or to a water treatment and disposal apparatus 244. A water transfer pump 242 may be used to pump the water through the system 200.

The system 200 may comprise or operate in conjunction with a control center 246 containing equipment for monitoring and/or controlling the system 200. For example, the control center 246 may comprise data acquisition and/or control equipment for monitoring and/or controlling the system 200. The control center 246 may be set in a non-hazardous area 248 apart from the hazardous well test area containing the other equipment of the well testing system 200. The control center 246 may contain or comprise a data processing system 249 for monitoring and/or controlling the system 200. Various types of information may be automatically acquired from sensors of the system 200 and then processed by the data processing system 249. The data processing system 249 may provide various functions, such as a sensor data display, video display, sensor or video information interpretation for quality-assurance and quality-control purposes, and/or data input devices for manual entry of various operational parameters and set-points.

The system 200 may be monitored during well testing operations to verify proper operation and facilitate control of the well testing operations. Such monitoring may include taking numerous measurements during a well test, examples of which can include choke manifold temperature and pressure (upstream and downstream), heat exchanger temperature and pressure, separator temperature and pressure (static and differential), oil flow rate and volume from the separator, water flow rate and volume from the separator, and fluid levels in tanks.

The system 200 may comprise or operate in conjunction with a gas monitoring system 128 operable to monitor various properties of a gas plume produced by the burning well fluids (e.g., oil and/or gas) during burning operations performed by one or more of the burning devices 226, 227. For example, the gas monitoring system 128 may be operable to monitor concentrations and/or rates at which various individual component gases within or forming the gas plume are produced (i.e., emitted) by the burning of the well fluids via one or more of the burning devices 226, 227. The gas monitoring system 128 may also or instead be operable to monitor the efficiency of burning (i.e., combustion efficiency) of the well fluids by one or more of the burning devices 226, 227. At least a portion of the gas monitoring system 128 may be located and/or operate in association with one or more of the burning devices 226, 227, such as may permit the gas monitoring system 128 to monitor the various properties of the gas plume. The data processing system 249 may operate in conjunction with the gas monitoring system 128 or the data processing system 249 may be or form a portion of the gas monitoring system 128. The data processing system 249 may be communicatively connected with the gas monitoring system 128 and operable to receive and process sensor data from the gas monitoring system 128.

The present disclosure is further directed to systems and methods (i.e., processes, operations, etc.) for measuring or otherwise monitoring various properties of a gas plume produced by the burning of a hydrocarbon effluent during burning operations performed at a worksite or facility by a burning device. The hydrocarbon effluent may comprise, for example, well fluids (e.g., oil and/or gas) that are burned at a wellsite by a burner or flare during well testing operations described above. The systems and methods according to one or more aspects of the present disclosure may be used to determine efficiency of the burning (i.e., combustion efficiency) of the hydrocarbon effluent via the burning device. The systems and methods according to one or more aspects of the present disclosure may also or instead be used to estimate, calculate, or otherwise determine, in real-time, flow rates of individual component gases within or forming the gas plume that are produced (i.e., emitted) by the burning (i.e., combusting) of the hydrocarbon effluent via the burning device. Such determinations may be based on the composition (e.g., chemical analysis) and flow rate (e.g., flow rate measurements) of the hydrocarbon effluent that is being transmitted to the burning device, as well as optical spectroscopy analysis of the gas plume that is being produced by the burning of the hydrocarbon effluent via the burning device.

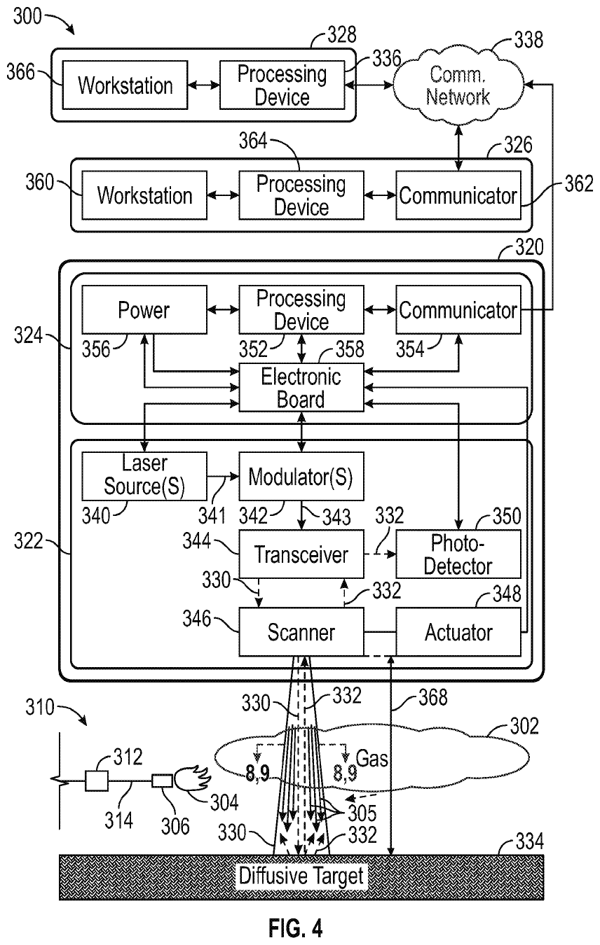

FIG. 4 shows an example implementation of a gas monitoring system 300 operable to determine various properties of a gas plume 302 produced by burning 304 of a hydrocarbon effluent being transmitted (i.e., flowing) to a burning device 306 at a hydrocarbon burning facility 310. The hydrocarbon burning facility 310 may be a hydrocarbon (e.g., oil and/or gas) producing facility (e.g., the marine environment 102 and/or the land environment 104 shown in FIG. 1) at which burning of at least a portion of the produced hydrocarbons is performed, such as during well testing operations and/or during hydrocarbon production operations. However, the gas monitoring system 300 may instead be utilized at or in association with other facilities, such as hydrocarbon distribution, processing, and/or refining facilities at which the burning of hydrocarbons is performed, but at which the hydrocarbons are not necessarily produced. If the hydrocarbon effluent is or comprises oil, the burning device 306 may be or comprise a burner operable to burn the oil. If the hydrocarbon effluent is or comprises a gas, the burning device 306 may be or comprise a flare. Thus, for the sake of clarity and ease of understanding, the term “burning device” herein is to be interpreted as either an oil-combusting burner or a gas-combusting flare.

The gas monitoring system 300 may comprise a flow rate sensor 312 fluidly or otherwise operatively connected along a fluid conduit 314 fluidly connecting a source (not shown) of the hydrocarbon effluent and the burning device 306. The flow rate sensor 312 may be operable to facilitate measuring or otherwise obtaining the volumetric and/or mass flow rate of the hydrocarbon effluent. The flow rate sensor 312 may be operable to output or otherwise facilitate flow rate data indicative of the flow rate of the hydrocarbon effluent within the fluid conduit 314. The flow rate sensor 312 may be an electrical flow rate sensor operable to output electrical flow rate data indicative of the measured flow rate. The flow rate sensor 312 may be a Coriolis flowmeter, a turbine flowmeter, or an acoustic flowmeter, among other examples.

The gas monitoring system 300 may further comprise a laser system 320 located in association with the hydrocarbon burning facility 310 or at the worksite comprising the hydrocarbon burning facility 310. The laser system 320 may be located and/or operate in association with the burning device 306, such that the gas plume 302 is within a field of view of the laser system 320 and, thus, permits the gas monitoring system 300 to monitor the properties of the gas plume 302. The laser system 320 may comprise a laser emission and detection system 322, such as may have one or more aspects in common with or similar to a laser system as described in PCT Patent Publication No. WO2021023971A1, the entirety of which is hereby incorporated herein by reference. The laser emission and detection system 322 is operable to emit one or more laser beams 330 that pass through the gas plume 302. The laser emission and detection system 322 may be operable to detect (i.e., measure) the intensity of a reflected (i.e., backscattered) portion (hereinafter “backscatter” or “reflection”) 332 of the laser beam(s) 330 that, after passing through the gas plume 302 and being backscattered by a diffusive target 334 (e.g., land surface, water surface, a building surface, a barrier, etc.), returns to the laser emission and detection system 322. The laser system 320 may further comprise a power and control system 324 operable to supply electrical power to and control the laser emission and detection system 322.

The gas monitoring system 300 may comprise a local data processing system 326 located in association with the hydrocarbon burning facility 310 or at the worksite comprising the hydrocarbon burning facility 310. The gas monitoring system 300 may comprise a remote data processing system 328 located at a remote location (i.e., a different worksite from the worksite at which the hydrocarbon burning facility 310 is located). The local data processing system 326 and the remote data processing system 328 may each be communicatively connected with the laser system 320 and operable to process (i.e., analyze) sensor data output by the laser system 320 to determine the properties of the gas plume 302. The local data processing system 326 and the remote data processing system 328 may each be communicatively connected with the laser system 320 via one or more communication networks 338 (e.g., the internet, a cellular communication network, a satellite communication network, a wide area network (WAN), a local area network (LAN), etc.).

The laser emission and detection system 322 and the power and control system 324 of the laser system 320 may be located in close association with each other and be electrically, communicatively, and/or physically connected with each other. For example, the systems 322, 324 may be or form at least a portion of the same device, assembly, or unit. Similarly, the local data processing system 326 may be located in close association with the laser system 320 and be electrically, communicatively, and/or physically connected with the laser system 320. For example, the laser system 320 and the local data processing system 326 may be or form at least a portion of the same device, assembly, or unit. In such implementations, communication between the laser system 320 and the local data processing system 326 may be performed via wired communication means. However, the local data processing system 326 may be electrically and/or communicatively connected with the laser system 320, but the local data processing system 326 may be or form at least a portion of a device, assembly, or unit that is separate from the laser system 320. In such implementations, the local data processing system 326 may be located at a distance (e.g., several meters to several hundred meters or more) from the laser system 320, and communication between the laser system 320 and the local data processing system 326 may be performed via wired or wireless communication means.

The laser emission and detection system 322 may comprise a laser source 340 (e.g., a distributed feedback (DFB) laser device, a semiconductor laser device, a diode laser device, a tuneable diode laser device, a narrow-linewidth laser device, an indium phosphide laser device, etc.) operable to emit a laser beam 341. The laser source 340 may be operable to tune (e.g., change or adjust) the wavelength of the laser beam 341, such as by changing the duration and/or magnitude of the electrical current input to the laser source 340 from the power and control system 324. The tuned laser beam 341 may be modulated, such as via a modulator 342 operable to modulate the continuous laser beam 341 into a random or quasi-random bit stream, to thereby impart a modulated signal to or within the laser beam 341. The modulated signal within a modulated laser beam 343 may be used for cross-correlating the laser beam 330 output by the laser emission and detection system 322 with corresponding backscatter 332.

As depicted in figures described below, the laser source 340 may comprise a plurality of laser sources 340 each operable to emit a corresponding laser beam 341. Thus, reference below to a laser beam 341 may also refer to, be applicable to, or be readily adapted for two or more laser beams 341. In implementations utilizing first and second (or more) laser beams 341, among others within the scope of the present disclosure, the modulator 342 may comprise a plurality of modulators 343 each operable to receive and modulate a corresponding laser beam 341 and output a modulated laser beam 343 according to a modulation scheme. For example, the at least one of the first and second laser beams 343 may comprise a corresponding modulated signal. The first and second laser beams 343 may be modulated according to first and second modulation schemes that are the same or different.

The tuned and modulated laser beam 343 may then be directed to a transceiver 344. The laser beams 341, 343 may be transmitted between the laser source 340, the modulator 342, and the transceiver 344 via corresponding fiber optic cables (not shown). The transceiver 344 may comprise a plurality of lenses and/or mirrors collectively operable to output a tuned and modulated laser beam 330 into free space. For example, the transceiver 344 may be operable to output the laser beam 330 into the free space extending between the transceiver 344 and a scanner 346, such as by focusing and directing the laser beam 330 to travel through such free space toward the scanner 346, which directs the laser beam 330 out of the laser system 320 in an intended direction.

The scanner 346 may be or comprise a mirror, a set of optical wedges, optical prisms, and/or other means operable to direct the laser beam 330 in an intended direction, such as along one or more paths 305 through a laser data acquisition space 303 (i.e., an air column) extending through or otherwise containing at least a portion of the gas plume 302 that is to be tested for one or more properties. The scanner 346 may be mechanically or otherwise operatively connected to an actuator 348 (e.g., an electric motor) operable to move (e.g., rotate) the scanner 346 to change the direction of the laser beam 330. The actuator 348 may repeatedly and continuously move (e.g., oscillate) the scanner 346 to repeatedly and continuously change direction of (i.e., scan) the laser beam 330 to thereby direct the laser beam 330 along a plurality of paths 305 through the space 303 extending through or otherwise containing the gas plume 302. The backscatter 332 of the laser beam 330 that passes through the space 303 may be received by the scanner 346 and directed to the transceiver 344, which may then direct the backscatter 332 to a photodetector 350 operable to detect (i.e., measure) intensity of the backscatter 332 (i.e., reflection).

The photodetector 350 may be operable to output or otherwise facilitate determining intensity data indicative of the intensity of the backscatter 332 received by the photodetector 350. The photodetector 350 may be or comprise one or more semiconductor-based photodetector devices, single-photon detector devices, single-photon avalanche diodes (SPADs), avalanche photodiodes (APDs), linear-mode APDs, silicon-based detector devices, and/or indium gallium arsenide-based detector devices. The photodetector 350 may be or comprise one or more complementary metal-oxide-semiconductor (CMOS) devices, in which at least a part of the photodetector 350 may be manufactured using a CMOS manufacturing process.

The power and control system 324 may comprise a processing device 352 (e.g., a computer, a programmable logic controller (PLC), etc.), a communication device 354, and a power source 356 (e.g., a power distribution device, an electrical amplifier, a battery, etc.). An electronics board 358 and/or other electrical circuitry may communicatively (i.e., electrically) connect the processing device 352 to the devices of the laser emission and detection system 322. The electronics board 358 may facilitate the transfer of data (e.g., sensor data, control commands, etc.) between the processing device 352 and one or more of the power source 356, the communication device 354, the laser source 340, the modulator 342, the transceiver 344, the photodetector 350, and the actuator 348. The electronics board 358 may also facilitate the transfer of electrical power from the power source 356 to one or more of the processing device 352, the communication device 354, the laser source 340, the modulator 342, the transceiver 344, the photodetector 350, and the actuator 348.

The local data processing system 326 may comprise a processing device 364 and a communication device 362. The remote data processing system 328 may comprise a processing device 336 and a communication device (not shown). The communication device 362 and the communication device of the remote data processing system 328 may facilitate the transmission of data (e.g., sensor data) from the processing device 352 of the laser system 320 to the processing devices 364, 336, respectively, and the transmission of data (e.g., control commands) from the processing devices 364, 336, respectively, to the processing device 352 of the laser system 320. The processing devices 336, 364 may be communicatively connected (e.g., via respective communication devices) with the flow rate sensor 312 and be operable to receive and process the flow rate data facilitated by the flow rate sensor 312. Each processing device 336, 352, 364 may be operable to receive, process, and output data to monitor operations of, and/or provide control to, one or more devices or portions of the gas monitoring system 300. Each processing device 336, 352, 364 may store executable program code, instructions, and/or operational parameters or set-points, including for implementing one or more methods (e.g., processes, operations, etc.) described herein. The processing devices 336, 352, 364 may collectively form a processing system operable to control or otherwise cause the gas monitoring system 300 to perform one or more methods described herein.

Each data processing system 326, 328 may comprise or be communicatively connected with a corresponding workstation 360, 366 usable by a human operator (e.g., rig personnel) to monitor and control various devices of the gas monitoring system 300, such as the flow rate sensor 312 and/or the devices of the laser system 320. Each control workstation 360, 366 may be communicatively connected with a corresponding processing device 364, 336. For example, each control workstation 360, 366 may be operable for entering or otherwise communicating control commands to a corresponding processing device 364, 336 by the human operator, and for displaying or otherwise communicating information from the corresponding processing device 364, 336 to the human operator. Each control workstation 360, 366 may comprise one or more input devices (e.g., a keyboard, a mouse, a joystick, a touchscreen, etc.) and one or more output devices (e.g., a video monitor, a touchscreen, a printer, audio speakers, etc.). Each control workstation 360, 366 may be communicatively connected with the processing device 352 via the communicative connection between the data processing systems 326, 328 and the laser system 320. The workstation 360 may be located within a monitoring and control center (e.g., a room, a cabin, a trailer, etc.) at the worksite comprising the hydrocarbon burning facility 310. The workstation 366 may be located within a monitoring and control center located a different worksite from the worksite at which the hydrocarbon burning facility 310 is located.

The processing device 352 may be operable to monitor and control operation of the laser system 320. For example, the processing device 352 may cause the power source 356 to output electrical power to the laser source 340 to cause the laser source 340 to emit the laser beam 341. The processing device 352 may cause the scanner 346 to direct the laser beam 330 along a path 305 through the space 303 containing the gas plume 302. The processing device 352 may cause the power source 356 to vary the electrical power supplied to the laser source 340 to thereby tune (i.e., vary, scan, sweep, etc.) the wavelength of the laser beam 330 around (or through) a wavelength corresponding to a spectral absorption line of a predetermined (i.e., target) gas. The processing device 352 may then receive the intensity data output by the photodetector 350 indicative of intensity of the backscatter 332 (i.e., the portion of the modulated laser beam 330 that has been backscattered by the diffusive target 334) to thereby measure intensity of the backscatter 332 after passing through the gas plume 302. The processing device 352 may instead cause the scanner 346 to direct the laser beam 330 along a plurality of paths 305 through the space 303 containing the gas plume 302. For each of the paths 305 through the space 303, the processing device 352 may tune the wavelength of the laser beam 330 around a wavelength corresponding to a spectral absorption line of a predetermined gas and receive the intensity data output by the photodetector 350 to thereby measure intensity of the backscatter 332 after passing through the gas plume 302. Predetermined (i.e., target) gases for which presence and properties in the gas plume 302 may be tested (i.e., for which the intensity of the backscatter 332 may be measured) may include, for example, CO, CO₂, CH₄, C₂H₂, C₂H₄, C₃H₆, NO, NO₂, NO₃, SO₂, and/or other gases that can be produced by burning (i.e., combusting) a hydrocarbon effluent.

The processing device 352 may also determine the length 368 of each path 305 between the laser emission and detection system 322 (e.g., the scanner 346) and the diffusive target 334, such as based on an amount of time that the laser beam 330 (and the backscatter 332) travels back and forth between the laser emission and detection system 322 and the diffusive target 334. For example, the random or quasi-random modulation may be utilized to cross-correlate the emitted laser 330 and the received backscatter 332. Thus, the processing device 352 may determine the length 368 of a path 305 through the gas plume 302 based on the amount of time between when the laser beam 330 comprising a modulated signal has been emitted by the laser source 340 and when the corresponding backscatter 332 comprising the modulated signal has been received by the photodetector 350. Accordingly, in some respects, the laser emission and detection system 322 may operate similar to a laser imaging, detection, and ranging (LIDAR) system.

FIG. 5 is a graph showing an example spectrogram 400 of a measured intensity 404 of an output signal 402 generated by the photodetector 350 measuring or otherwise based on the backscatter 332 of the laser system 320 shown in FIG. 4, with respect to wavelength of the laser beam 330 (and the backscatter 332). The intensity 404 is or comprises magnitude of intensity data output by the photodetector 350 as the wavelength of the laser beam 330 is tuned (i.e., swept) through a range of wavelengths 406 including a predetermined wavelength 408 corresponding to a spectral absorption line of a predetermined (i.e., target) gas that may form or be within the gas plume 302. As the wavelength of the laser beam 330 approaches the wavelength 408 corresponding to the spectral absorption line of the predetermined gas, the intensity 404 may decrease by a measurable amount 410 based on the amount of such predetermined gas existing in the gas plume 302. Thus, if the intensity 404 experiences an attenuation 410 at a wavelength 408 corresponding to a spectral absorption line of a predetermined gas, the presence of such predetermined gas is confirmed as a component gas of the gas plume 302. Depending on the wavelength 406 of the laser beam 330, the intensity 404 of the signal 402 output by the photodetector 350 may be more or less attenuated and, thus, indicative of concentration of the component gas within the gas plume 302, such as based on the Beer-Lambert Law.

The photodetector 350 may be operable to output intensity data for a wide range of laser wavelengths. The spectrogram 400 may thus show a wide range of intensity 404 and a plurality of intensity drops 410, each associated with a corresponding spectral absorption line of a corresponding predetermined gas. The measured intensity 404 for each wavelength, or at least at the wavelength 408, may be recorded by one or more of the processing devices 336, 352, 364. Such process may be repeated for a predetermined number of different paths 305 at different locations through the space 303 containing the gas plume 302. The different wavelengths of the emitted laser beams 330 and the resulting different intensities of the detected backscatter 332 at different integration times may then be utilized to generate the spectrogram 400.

For example, a human operator or one or more of the processing devices 336, 352, 364 may select one or more predetermined gases intended to be tested for or monitored by the gas monitoring system 300, define a space 303 containing at least a portion of the gas plume 302, and define a scheme (e.g., pattern or order) for scanning the space 303 with the laser beam 330 along a plurality of paths 305 though the space 303. For each of the defined paths 305 through the space 303, the processing device 352 may then cause the laser beam 330 to be tuned around a wavelength corresponding to a spectral absorption line of the one or more predetermined gases and cause the photodetector 350 to measure intensity of the backscatter 332.

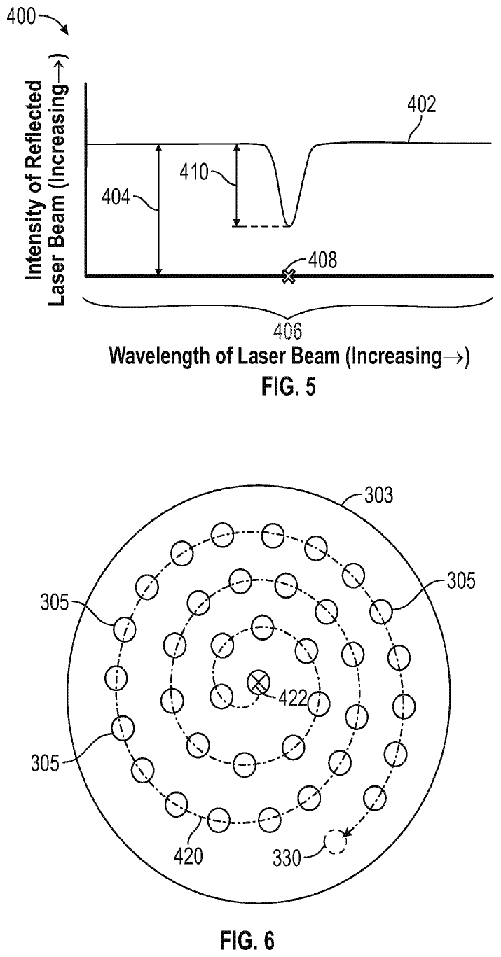

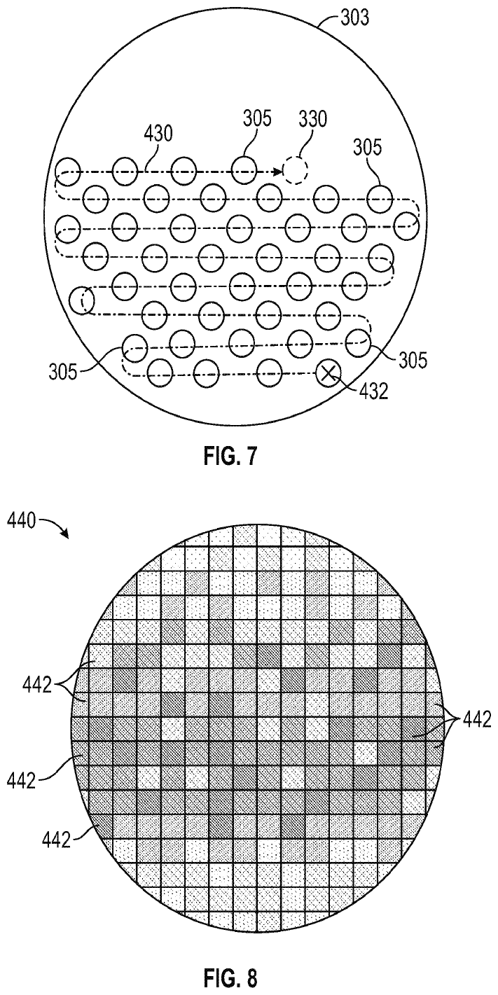

FIGS. 6 and 7 are schematic views of different schemes for scanning the space 303 containing at least a portion of the gas plume 302 shown in FIG. 4 with the laser beam 330 thereby permitting the laser beam 330 to pass the space 303 extending through the gas plume 302 along a plurality of paths 305 though the space 303. FIG. 6 shows a spiral scanning scheme (or scanning path) 420, wherein the processing device 352 causes the scanner 346 to direct the laser beam 330 along a path 305 extending though the center 422 of the defined space 303 and then progressively directing the laser beam 330 in a spiral manner around the center 422 until the scanner 346 causes the laser beam 330 to cover (i.e., pass through) the entire defined space 303 or predetermined locations of the paths 305 through the space 303. At each location of the paths 305, the processing device 352 may cause the laser beam 330 to be tuned to different wavelengths in a range around a predetermined wavelength corresponding to a spectral absorption line of the predetermined gas, and may cause the photodetector 350 to measure intensity of the backscatter 332. FIG. 7 shows an alternating (e.g., zig-zag) linear scanning scheme 430, wherein the processing device 352 causes the scanner 346 to direct the laser beam 330 along a path 305 extending though the defined space 303 on a side 432 (i.e., a lateral position) of the space 303 and then progressively directs the laser beam 330 in an alternating linear manner toward the opposing side of the space 303 until the scanner 346 causes the laser beam 330 to cover (i.e., pass through) the entire defined space 303 or predetermined locations of the paths 305 through the space 303. Although FIGS. 6 and 7 show the spaces 303 having a circular cross-section, the spaces 303 may be defined as having other cross-sectional geometries, including triangular, square, rectangular, or elliptical geometries, among other examples. The scanning through the space 303 may also have scanning paths other than as shown in FIGS. 6 and 7. For example, the laser beam 330 may be directed along a simple circular scanning path, or along a random scanning path in which the laser paths 305 extend through the defined space 303 at a random order and/or at random locations. However, scanning through the space 303 may not occur. For example, the laser beam 330 may be directed through the space 303 extending through the gas plume 302 along a single path 305.

The processing device 352 may be further operable to determine (e.g., measure, calculate, estimate, etc.) the concentration of a component gas within or forming the gas plume 302 along each of the paths 305 between the laser emission and detection system 322 and the diffusive target 334 based on the measured intensity 404 of the backscatter 332 of the modulated laser beam 330 along each of the paths 305. Because the concentration of the component gas is determined for an entire path 305 having a length 368, such concentration may be referred to as a “concentration path length,” which may comprise units of concentration (e.g., expressed in units of parts per million (PPM)) multiplied by the path length 368 (e.g., expressed in units of meters (M)). Thus, a spectrogram (e.g., the spectrogram 400) generated by the processing device 352 may be indicative of a concentration path length of the component gas along each of the paths 305 between the laser emission and detection system 322 and the diffusive target 334.

FIG. 8 is an example concentration path length map 440 indicative of concentration path lengths of a component gas within a defined space 303 along a plurality of paths 305 between the laser emission and detection system 322 and the diffusive target 334. The concentration path length map 440 may be or comprise a discretization grid comprising a plurality of vertical columns and horizontal rows of discretized elements referred to as pixels 440. Each pixel 440 may be indicative of or otherwise based on concentration path length data, and each concentration path length data point may be indicative of a determined concentration path length measurement of the component gas within the defined space 303. The concentration path length map 440 may thus comprise a geometry corresponding to a cross-section of the defined space 303 scanned by the laser beam 330 of the laser system 320. Fach pixel 442 may correspond to a path 305 through the space 303 containing the gas plume 302, and may comprise a different color and/or brightness indicative of or otherwise based on a concentration path length along the corresponding path 305. Each pixel 442 may instead correspond to a plurality of paths 305 through the space 303 (and corresponding concentration path lengths) containing the gas plume 302, and may comprise a different color and/or brightness indicative of an average, highest, or lowest concentration path length along the corresponding paths 305. A concentration path length map (e.g., map 440) may be generated by one or more of the processing devices 336, 352, 364 based on concentration path lengths and one or more equations, algorithms, and/or computer program code described herein.

The gas monitoring system 300 may be operable to test for and determine a concentration path length of a predetermined gas, such as CO, CO₂, CH₄, C₂H₂, C₂H₄, C₃H₆, NO, NO₂, NO₃, SO₂, and/or other gases that can be produced by burning a hydrocarbon effluent. Because each predetermined gas possesses its own spectral properties and, thus, corresponds to a different spectral absorption line, the gas monitoring system 300 may be operated to determine a concentration path length along each path 305 for each predetermined gas.

For example, the laser emission and detection system 322 may comprise a single laser source 340 that may tune the wavelength of the laser beam 330 (i.e., the laser beam 341) through a range of wavelengths around and including spectral absorption lines of two or more predetermined gases. Thus, for each path 305, the wavelength of the laser beam 330 emitted by the laser source 340 may be swept (continuously, incrementally, or otherwise) through a range of wavelengths around and including the spectral absorption line of a first predetermined gas, and then the wavelength of the laser beam 330 may be swept through another range of wavelengths around and including the spectral absorption line of a second predetermined gas. The wavelength of the laser beam 330 may also be swept through other ranges of wavelengths around and including spectral absorption lines of other predetermined gases. Each laser beam 330 (i.e., intermediate laser beam 343) may be transmitted through the transceiver 344 and directed toward the scanner 346, which may direct the laser beam 330 along one or more paths 305 extending through the gas plume 302, as described herein. The photodetector 350 may then receive each corresponding backscatter 332, one at a time, and output corresponding intensity data to the processing device 352, which may then determine a concentration path length for each predetermined gas.

However, the laser system 320 may instead comprise a laser emission and detection system 322 comprising a plurality (e.g., two, three, four, or more) of dedicated devices or device sets, each operable to tune the wavelength of a corresponding laser beam 330 through a corresponding range of wavelengths around and including a spectral absorption line of a corresponding predetermined gas. For example, instead of using the same laser source 340 to tune the wavelength of the laser beam 330 through a range of wavelengths around and including spectral absorption lines of two or more predetermined gases, the laser emission and detection system 322 may comprise a plurality (e.g., two, three, four, or more) of laser sources 340 each operable to tune the wavelength of a corresponding laser beam 330 through a range of wavelengths around and including a spectral absorption line of a single predetermined gas (or, in some implementations, more than one predetermined gas when two or more predetermined gases have close absorption lines).

The laser emission and detection system 322 may also comprise a corresponding modulator 342 for each laser source 340, such as may permit each modulator 342 to modulate a corresponding tuned laser beam 330 (i.e., an intermediate laser beam 341) into a random or quasi-random bit stream, to thereby impart a modulated signal to or within the tuned laser beam 330 and, thus, permit cross-correlation (i.e., differentiation) of the backscatter 332 received by the photodetector 350 and distinguishing the backscatter from noise. The tuned and modulated laser beams 330 (i.e., laser beams 343) may be simultaneously or sequentially transmitted through the transceiver 344 and directed toward the scanner 346, which may simultaneously direct the laser beams 330 along one or more paths 305 extending through the gas plume 302, as described herein. However, the scanner 346 may instead be controlled independently to scan a corresponding laser beam 330 along one or more paths 305 extending through the gas plume 302. A single or plurality of photodetectors 350 may receive the reflections 332 and output the intensity data to the processing device 352. For example, a single photodetector 350 may receive a plurality of backscatters 332 and output the resulting intensity data to the processing device 352, which may then differentiate between each backscatter 332 (and the intensity data) based on the modulated signal associated with each laser beam 330 and, thus, permit the processing device 352 to determine a concentration path length for each predetermined gas.

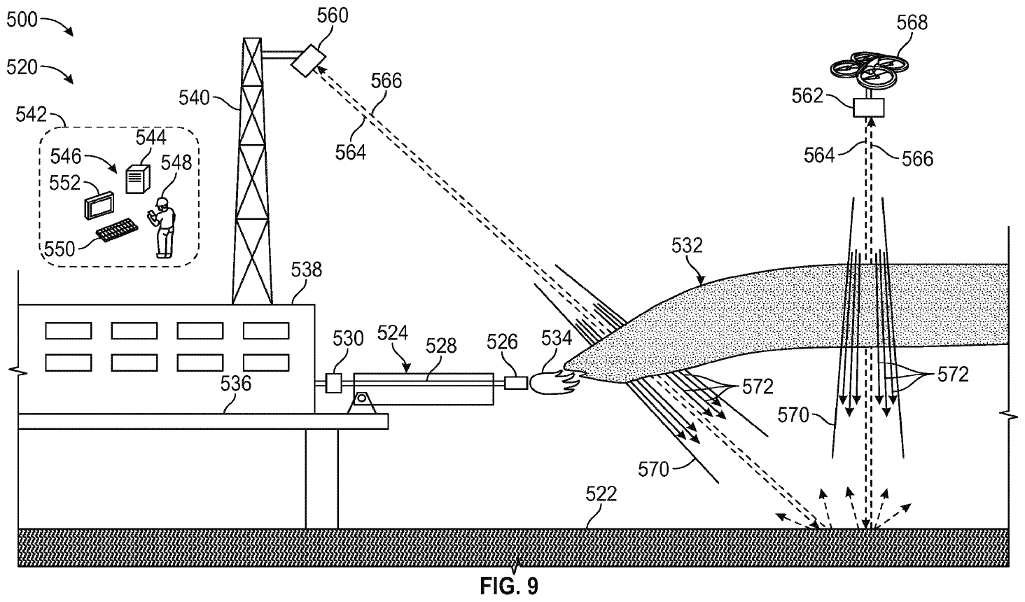

FIG. 9 is a schematic view of a gas monitoring system 500 implemented in a marine environment according to one or more aspects of the present disclosure. However, one or more of the aspects described below may also be applicable or readily adaptable for environments on land instead of marine environments. The gas monitoring system 500 comprises features and modes of operation of the gas monitoring system 300 shown in FIG. 4 and described above.

The marine environment comprises an offshore oil and gas well drilling and production rig 520 located above a water surface 522. The rig 520 comprises a burner boom 524 comprising a burning device 526 and a fluid conduit 528 fluidly connecting a source (not shown) of a hydrocarbon effluent and the burning device 526. Burning 534 of the hydrocarbon effluent at the burning device 526 may produce a gas plume 532 of pollutant gases dispersed into the ambient atmosphere. The rig 520 may further comprise a rig platform or floor 536, a main complex 538, and a support structure 540 extending upward from the main complex 538. The main complex 538 or other portion of the rig 520 may comprise a monitoring and control center 542 (e.g., a room, a cabin, etc.) comprising a processing device 544 and a monitoring and control workstation 546 usable by a human operator 548 (e.g., rig personnel) to monitor and control various devices or portions of the rig 520 and the gas monitoring system 500. The control workstation 546 may be communicatively connected with the processing device 544. The control workstation 546 may be operable for entering or otherwise communicating control commands to the processing device 544 by the human operator 548, as well as for displaying or otherwise communicating information from the processing device 544 to the human operator 548. The control workstation 546 may comprise one or more input devices 550 (e.g., a keyboard, a mouse, a joystick, a touchscreen, etc.) and one or more output devices 552 (e.g., a video monitor, a touchscreen, a printer, audio speakers, etc.).

The gas monitoring system 500 may comprise a flow rate sensor 530 fluidly or otherwise operatively connected along the fluid conduit 528. The flow rate sensor 530 may be operable to measure volumetric and/or mass flow rate of the effluent being transferred to the burning device 526 via the fluid conduit 528. The processing device 544 may be communicatively connected with the flow rate sensor 530 and operable to receive and process the flow rate data facilitated by the flow rate sensor 530. The gas monitoring system 500 may comprise one or more laser systems 560, 562 each operable to emit one or more laser beams 564 through the gas plume 532 and to measure intensity of a backscatter 566 of each laser beam 564 by the water surface 522. Each laser system 560, 562 may tune its laser beam(s) 564 around a wavelength corresponding to a spectral absorption line of a predetermined gas. Each laser system 560, 562 may be operable to repeatedly and continuously change direction of (i.e., scan) its laser beam 564 to direct the laser beam 564 along a plurality of paths 572 through a space 570 containing at least a portion of the gas plume 532. Thus, each laser system 560, 562 may comprise one or more features and/or modes of operation of the laser system 320 shown in FIG. 4 and described above. The laser system 560 may be connected to or otherwise supported by the support structure 540 above or in view of the burning device 526 and, thus, in view of the gas plume 532 produced by the burning of the hydrocarbon effluent by the burning device 526. The laser system 562 may be connected to and flown over or otherwise toward the gas plume 532 via a flying device 568 (e.g., an aerial drone).

Each laser system 560, 562 may be programmed with a scheme for scanning the space 570 with the laser beam 564. Because the location of the laser system 560 is fixed, the laser system 560 may be programmed just to change direction of its laser beam 564 to direct the laser beam 564 along a predetermined scanning path through the corresponding space 570. However, because the laser system 562 is mobile, the flying device 568 may be programmed to fly in a predetermined pattern and the laser system 562 may be programmed to change the direction of its laser beam 564, such as to collectively direct the laser beam 564 along a predetermined scanning path through the corresponding space 570.

The processing device 544 may comprise one or more features and/or modes of operation of the processing device 336 of the remote data processing system 328 and/or the processing device 364 of the local data processing system 326 shown in FIG. 4 and described above. For example, the processing device 544 may be communicatively connected with each laser system 560, 562 and may be further operable to receive and analyze intensity data output by each laser system 560, 562 indicative of the intensity of a corresponding backscatter 566. Based on the intensity data, the processing device 544 may determine (i.e., test) which predetermined gas is present in the gas plume 532 (thereby confirming such predetermined gas as a component gas forming the gas plume 532) and determine a concentration path length of each component gas. The processing device 544 may then determine a rate of emission of each component gas produced by burning of the hydrocarbon effluent and/or combustion efficiency of the hydrocarbon effluent during the burning operations, as described herein.

A gas monitoring system (e.g., the gas monitoring system 300, 500) according to one or more aspects of the present disclosure may be further operable to determine (e.g., calculate, estimate, etc.) various properties of a gas plume, such as a rate of emission (i.e., flow rate) of each component gas within or forming the gas plume and/or combustion efficiency of the hydrocarbon effluent during the burning operations. The rate of emission of each component gas may be determined based on knowledge of the composition and flow rate of the hydrocarbon effluent that is being transmitted to a burning device, as well as a concentration path length of each component gas detected in the gas plume relative to concentration of CO₂ gas within the gas plume.

Determination of the rate of emission of each component gas may be based on chemical analyses and assumptions related to the hydrocarbon effluent that is being burned. For example, oil and gas produced from a well can be generally described by the formula C_{X}H_{2(X+1)}, yielding Equation (1) set forth below:

$${M}_{oil} = xMc + 2 ( x + 1 ) {M}_{H} \qquad ( 1 )$$

where Mₒᵢₗ is the molar mass of the oil in grams per mole (g/mol), M_{C}=12 g/mol, and M_{H}=1 g/mol. Equation (1) may apply, for example, to saturated hydrocarbon effluents (e.g., no cycles or non-single bonds) and/or other hydrocarbon effluents.

A perfect combustion of oil can be represented by Equation (2) set forth below:

$${C}_{x} {H}_{2 ( x + 1 )} + \frac{3 x + 1}{2} {O}_{2} \to {x CO}_{2} + ( x + 1 ) {H}_{2} O \qquad ( 2 )$$

Molar flow rate of oil burned by the burning device can be expressed as set forth below in Equation (3):

$${Qm}_{oil} = \frac{{ρ}_{oil} \cdot {Q}_{oil}}{{M}_{oil} \cdot {10}^{- 3}} [ mol / s ] \qquad ( 3 )$$

where ρₒᵢₗ is the of density oil in kg/m³, Qₒᵢₗ is the volumetric flow rate of oil in m³/s, and Mₒᵢₗ is the molar mass of oil in g/mol.

Therefore, molar flow rate of CO₂ gas produced can be expressed as set forth below in Equation (4):

$${Qm}_{{CO}_{2}} = x \cdot {Qm}_{oil} [ mol / s ] \qquad ( 4 )$$

Volumetric flow rate of CO₂ gas can then be expressed as set forth below in Equation (5):

$${Q}_{{CO}_{2}} = x \frac{{ρ}_{oil} {Q}_{oil}}{{M}_{oil}} \cdot {V}_{m} [ {m}^{3} / s ] \qquad ( 5 )$$

where Vₘ is molar volume of 22.414 L/mol, and mass flow rate of CO₂ gas can be expressed as set forth below in Equation (6):

$${Q}_{{CO}_{2}} = x \frac{{ρ}_{oil} \cdot {Q}_{oil} \cdot {M}_{{CO}_{2}}}{{M}_{oil}} [ kg / s ] \qquad ( 6 )$$

where M_{CO}_{₂} is the molar mass of CO₂ in g/mol.

In practice, combustion of oil is not perfect, resulting in other component gases, such as CO, CH₄, NO, NO₂, NO₃, and SO₂, being produced by the burning operations and, thus, contained within the gas plume 302. Because the amount of carbon before and after combustion remains constant (i.e., is conserved), gases containing carbon produced during combustion can be calculated using the conservation of carbon as set forth below in Equation (7):

$${Qm}_{C} = {Qm}_{{CO}_{2}} + {Qm}_{{CH}_{4}} + {Qm}_{CO} \qquad ( 7 )$$

where Qm_{C} [mol/s] is the molar flow rate of carbon, which is equal to molar flow rate of CO₂ produced during perfect combustion.

Assuming that the concentration of each of the component gases forming the gas plume 302 is representative of a relative molar flow rate of such gases, the conservation of carbon can be rewritten as set forth below in Equation (8):

$${Qm}_{C} = {Qm}_{{CO}_{2}} + \frac{[ {CH}_{4} ]}{[ {CO}_{2} ]} {Qm}_{{CO}_{2}} + \frac{[ CO ]}{[ {CO}_{2} ]} Q {m}_{{CO}_{2}} \qquad ( 8 )$$

where [CH₄], [CO], and [CO₂] are concentrations of the component gases CH₄, CO, and CO₂, respectively, in the gas plume 302. The concentrations of the component gases forming the gas plume may be expressed in units of PPM.

The concentration of each component gas, as expressed by corresponding symbols within brackets “[ ]” in various equations listed herein, may be estimated or substituted with the concentration path length of that component gas. The concentration path length of each component gas may be expressed in units of PPM·M. The concentration path length of each component gas may be determined by testing (via a laser device) the gas plume for each predetermined gas that can exist in the gas plume via the methods (or processes) described herein. The concentration path length determined for each component gas found in the gas plume may, thus, be used as the concentration path length of each component gas in the Equations (8)-(16) listed herein.

Molar flow rate of each component gas containing carbon may thus be expressed as set forth below in Equations (9)-(11):

$${Qm}_{{CO}_{2}} = \frac{{Qm}_{C}}{1 + \frac{[ {CH}_{4} ]}{[ {CO}_{2} ]} + \frac{[ CO ]}{[ {CO}_{2} ]}} \qquad ( 9 )$$

$${Qm}_{{CH}_{4}} = \frac{[ {CH}_{4} ]}{[ {CO}_{2} ]} {Qm}_{{CO}_{2}} \qquad ( 10 )$$

$${Qm}_{CO} = \frac{[ CO ]}{[ {CO}_{2} ]} {Qm}_{{CO}_{2}} \qquad ( 11 )$$

where Qm_{CO}_{₂}, Qm_{CH}_{₄}, and Qm_{CO} are the molar flow rates of the gases CO₂, CH₄, and CO, respectively, produced by the burning of the hydrocarbon effluent. Molar flow rates for other component gases (e.g., C₂H₂, C₂H₄, C₃H₆, etc.) that can be produced by the burning of the hydrocarbon effluent may be similarly determined by determining a ratio (i.e., a quotient) of the concentration path length of a component gas to the concentration path length of CO₂ and multiplying such ratio by the molar flow rate of CO₂.

The molar flow rate of each component gas not containing carbon may be similarly expressed as set forth below in Equations (12)-(15):

$${Qm}_{NO} = \frac{[ NO ]}{[ {CO}_{2} ]} {Qm}_{{CO}_{2}} \qquad ( 12 )$$

$${Qm}_{{NO}_{2}} = \frac{[ {NO}_{2} ]}{[ {CO}_{2} ]} {Qm}_{{CO}_{2}} \qquad ( 13 )$$

$${Qm}_{{NO}_{3}} = \frac{[ {NO}_{3} ]}{[ {CO}_{2} ]} {Qm}_{{CO}_{2}} \qquad ( 14 )$$

$${Qm}_{{SO}_{2}} = \frac{[ {SO}_{2} ]}{[ {CO}_{2} ]} {Qm}_{{CO}_{2}} \qquad ( 15 )$$

where Qm_{NO}, Qm_{NO}_{₂}, Qm_{NO}_{₃}, and Qm_{SO}_{₂} are the molar flow rates of the gases NO, NO₂, NO₃, and SO₂, respectively, produced by the burning of the hydrocarbon effluent.

The molar flow rate Equations (9)-(15) listed above may be utilized to determine (e.g., calculate, estimate, etc.) the rate of emission (i.e., flow rate) of each component gas during the burning operations. Although a plurality of concentration path lengths may be determined for each component gas in the gas plume, a single representative concentration path length for each component gas in the gas plume may first be determined for use in the molar flow rate Equations (9)-(15) by applying one or more statistical analyses to the determined concentration path lengths. For example, the representative concentration path length of each component gas may be determined by calculating a mean (or average) concentration path length for the plurality of paths through the space extending though the gas plume for each component gas that is detected (i.e., determined to have a concentration path length) in the gas plume. The mean concentration path length may instead be determined for some of the laser paths through the space extending though the gas plume, such as a restricted region of the space where the gas plume comprises higher concentrations of a component gas. After the representative concentration of each component gas in the gas plume is determined, one or more processing devices (e.g., processing devices 336, 352, 364) may calculate a ratio (i.e., a quotient) of a concentration path length of each component gas to a concentration path length of CO₂ gas. The calculated ratios may then be used in one or more of the Equations (8)-(15) listed above to determine a molar flow rate of each component gas.

One or more of the processing devices may then determine the flow rate at which each component gas is being produced by the burning of the hydrocarbon effluent via the burning device based on the molar flow rate Equations (9)-(15) listed above for each component gas and knowledge of the composition and flow rate of the hydrocarbon effluent that is being transmitted to the burning device. The composition of the hydrocarbon effluent may include the molar weight and the density of the hydrocarbon effluent. Data indicative of the composition of the hydrocarbon effluent may be entered to one or more of the processing devices by a human operator via workstations. Data indicative of the flow rate of the hydrocarbon effluent (Qₒᵢₗ in Equations (3) and (5)) may be facilitated by a flow sensor and communicated to one or more of the processing devices or entered to one or more of the processing devices by a human operator via the workstations. After one or more of the processing devices determines the molar flow rates of each component gas using the molar flow rate Equations (9)-(15), one or more of the processing devices may then convert such molar flow rates to mass and/or volumetric flow rate of each component gas using or otherwise based on the molar weight of the effluent, the density of the hydrocarbon effluent, and the flow rate of the hydrocarbon effluent.

The present disclosure also introduces one or more aspects pertaining to combustion efficiency (CE). CE is an example answer product that can be determined utilizing the measurements described herein, particularly in the absence of flowrate and molecular mass measurements. CE can be utilized to quantify the efficiency of the burning process and may provide feedback to act directly on the burning process. CE can be expressed as set forth below in Equation (16):

$$CE = [ {CO}_{2} ] / ( [ {CO}_{2} ] + [ UH ] + [ CO ] ) \qquad ( 16 )$$

where UH is unburnt hydrocarbon and the brackets [ ] indicate concentration in units of PPM.

This quantity estimates the efficiency of the burning of the hydrocarbon effluent via the burning device 306. That is, CE is ideally 100%, meaning that each atom of carbon in the hydrocarbon is converted into CO₂. Utilizing one or more aspects introduced in the present disclosure, the concentration in units of PPM may be replaced by concentration path length inunits of PPM·M, because the gases are each measured along the same path. UH can be replaced by CH₄ because the amounts of unburned hydrocarbons of other types are generally low relative to the amount of unburned CH₄, especially in natural gas. Accordingly, measuring CO₂, CO, and CH₄ utilizing the devices and processes described above permits determining CE. A simplification would be to measure just CO₂ and CH₄ to get an estimation of CE with a cheaper system, because the amount of CO is generally low relative to the amount of unburned CH₄, especially in natural gas. In this case, combustion efficiency may be determined as set forth below in Equation (17).

$$CE = [ {CO}_{2} ] / ( [ {CO}_{2} ] + [ {CH}_{4} ] ) \qquad ( 17 )$$

Equation (17) may be rewritten in percentage terms as set forth below in Equation (18).

$$CE ( \% ) = 100 [ {CO}_{2} ] / ( [ {CO}_{2} ] + [ {CH}_{4} ] ) \qquad ( 18 )$$

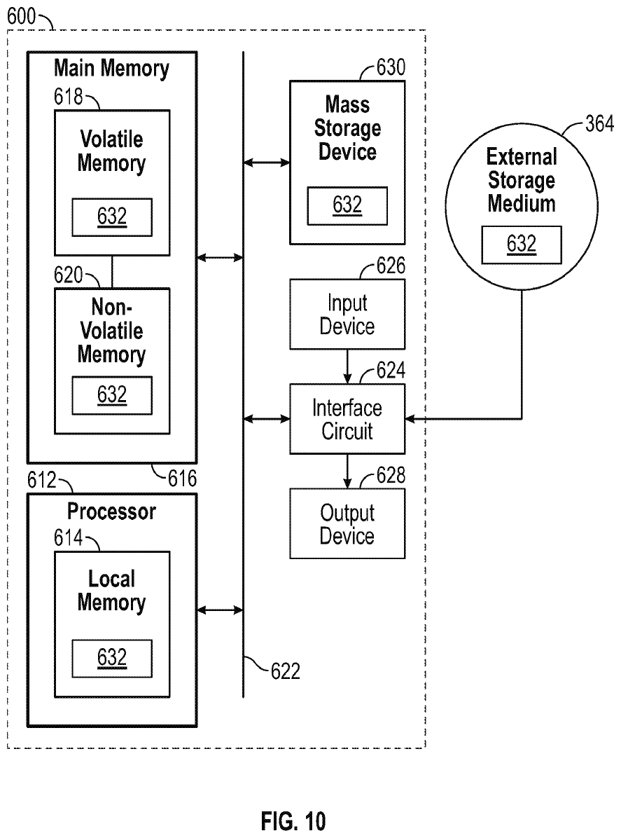

FIG. 10 is a schematic view of at least a portion of an example implementation of a processing device (or system) 600 according to one or more aspects of the present disclosure. The processing device 600 may be or form at least a portion of one or more control devices and/or other electronic devices shown in one or more of the FIGS. 1-9. Accordingly, the following description refers to FIGS. 1-10, collectively.

The processing device 600 may be or comprise, for example, one or more processors, controllers, special-purpose computing devices, PCs (e.g., desktop, laptop, and/or tablet computers), personal digital assistants, smartphones, IPCs, PLCs, servers, internet appliances, and/or other types of computing devices. One or more instances of the processing device 600 may be or form at least a portion of the systems 111, 128, 249, 300, 500 or other monitoring and/or control system within the scope of the present disclosure. For example, one or more instances of the processing device 600 may be or form at least a portion of the control workstations 360, 366, 546 and/or the processing devices 336, 352, 364, 544. Although it is possible that the entirety of the processing device 600 is implemented within one device, it is also contemplated that one or more components or functions of the processing device 600 may be implemented across multiple devices, some or an entirety of which may be at a site and/or remote from the site.

The processing device 600 may comprise a processor 612, such as a general-purpose programmable processor. The processor 612 may comprise a local memory 614 and may execute machine-readable and executable program code instructions 632 (i.e., computer program code) present in the local memory 614 and/or other memory device. The processor 612 may execute, among other things, the program code instructions 632 and/or other instructions and/or programs to implement the example methods and/or operations described herein. For example, the program code instructions 632, when executed by the processor 612 of the processing device 600, may cause one or more portions or pieces of the systems 111, 128, 249, 300, 500 within the scope of the present disclosure to perform the example methods and/or operations described herein.

The processor 612 may be, comprise, or be implemented by one or more processors of various types suitable to the local application environment, and may include one or more of general-purpose computers, special-purpose computers, microprocessors, digital signal processors (DSPs), field-programmable gate arrays (FPGAs), application-specific integrated circuits (ASICs), and processors based on a multi-core processor architecture, as non-limiting examples. Examples of the processor 612 include one or more INTEL microprocessors, microcontrollers from the ARM and/or PICO families of microcontrollers, embedded soft/hard processors in one or more FPGAs.

The processor 612 may be in communication with a main memory 616, such as may include a volatile memory 618 and a non-volatile memory 620, perhaps via a bus 622 and/or other communication means. The volatile memory 618 may be, comprise, or be implemented by random-access memory (RAM), static RAM (SRAM), dynamic RAM (DRAM), synchronous DRAM (SDRAM), RAMBUS DRAM (RDRAM), and/or other types of RAM devices. The non-volatile memory 620 may be, comprise, or be implemented by read-only memory, flash memory, and/or other types of memory devices. One or more memory controllers (not shown) may control access to the volatile memory 618 and/or non-volatile memory 620.

The processing device 600 may also comprise an interface circuit 624, which is in communication with the processor 612, such as via the bus 622. The interface circuit 624 may be, comprise, or be implemented by various types of standard interfaces, such as an Ethernet interface, a universal serial bus (USB), a third-generation input/output (3GIO) interface, a wireless interface, a cellular interface, and/or a satellite interface, among others. The interface circuit 624 may comprise a graphics driver card. The interface circuit 624 may comprise a communication device, such as a modem or network interface card to facilitate exchange of data with external computing devices via a network (e.g., Ethernet connection, digital subscriber line (DSL), telephone line, coaxial cable, cellular telephone system, satellite, etc.).

The processing device 600 may be in communication with various sensors, video cameras, actuators, processing devices, control devices, and other devices of the systems 111, 128, 249, 300, 500 via the interface circuit 624. The interface circuit 624 can facilitate communications between the processing device 600 and one or more devices by utilizing one or more communication protocols, such as an Ethernet-based network protocol (such as ProfiNET, OPC, OPC/UA, Modbus TCP/IP, EtherCAT, UDP multicast, Siemens S7 communication, or the like), a proprietary communication protocol, and/or other communication protocol.

One or more input devices 626 may also be connected to the interface circuit 624. The input devices 626 may permit rig personnel to enter the program code instructions 632, which may be or comprise control data, operational parameters, operational set-points, and/or composition (e.g., molar weigh, density, etc.) of various hydrocarbon effluents. The program code instructions 632 may further comprise modeling or predictive routines, equations, algorithms, processes, applications, and/or other programs operable to perform example methods and/or operations described herein. The input devices 626 may be, comprise, or be implemented by a keyboard, a mouse, a joystick, a touchscreen, a track-pad, a trackball, an isopoint, and/or a voice recognition system, among other examples. One or more output devices 628 may also be connected to the interface circuit 624. The output devices 628 may permit for visualization or other sensory perception of various data, such as sensor data, status data, and/or other example data. The output devices 628 may be, comprise, or be implemented by video output devices (e.g., an LCD, an LED display, a CRT display, a touchscreen, etc.), printers, and/or speakers, among other examples. The one or more input devices 626 and the one or more output devices 628 connected to the interface circuit 624 may, at least in part, facilitate the HMIs described herein.

The processing device 600 may comprise a mass storage device 630 for storing data and program code instructions 632. The mass storage device 630 may be connected to the processor 612, such as via the bus 622. The mass storage device 630 may be or comprise a tangible, non-transitory storage medium, such as a floppy disk drive, a hard disk drive, a compact disk (CD) drive, and/or digital versatile disk (DVD) drive, among other examples. The processing device 600 may be communicatively connected with an external storage medium 634 via the interface circuit 624. The external storage medium 634 may be or comprise a removable storage medium (e.g., a CD or DVD), such as may be operable to store data and program code instructions 632.

As described above, the program code instructions 632 may be stored in the mass storage device 630, the main memory 616, the local memory 614, and/or the removable storage medium 634. Thus, the processing device 600 may be implemented in accordance with hardware (perhaps implemented in one or more chips including an integrated circuit, such as an ASIC), or may be implemented as software or firmware for execution by the processor 612. In the case of firmware or software, the implementation may be provided as a computer program product including a non-transitory, computer-readable medium or storage structure embodying computer program code instructions 632 (i.e., software or firmware) thereon for execution by the processor 612. The program code instructions 632 may include program instructions or computer program code that, when executed by the processor 612, may perform and/or cause performance of example methods, processes, and/or operations described herein.

The present disclosure is further directed to example operations, processes, workflows, algorithms, and other methods for monitoring and controlling devices of the systems 111, 128, 249, 300, 500. The example methods may be performed utilizing or otherwise in conjunction with one or more implementations of one or more instances of one or more components of the apparatus shown in one or more of FIGS. 1-10 and/or otherwise within the scope of the present disclosure. For example, the example methods may be at least partially performed (and/or caused to be performed) by a processing device, such as the processing device 600, executing program code instructions according to one or more aspects of the present disclosure. Thus, the present disclosure is also directed to a non-transitory, computer-readable medium comprising computer program code that, when executed by the processing device, may cause such processing device to perform the example methods described herein. The methods may also or instead be at least partially performed (or be caused to be performed) by a human operator (e.g., rig personnel) utilizing one or more implementations of one or more instances of one or more components of the apparatus shown in one or more of FIGS. 1-10 and/or otherwise within the scope of the present disclosure.

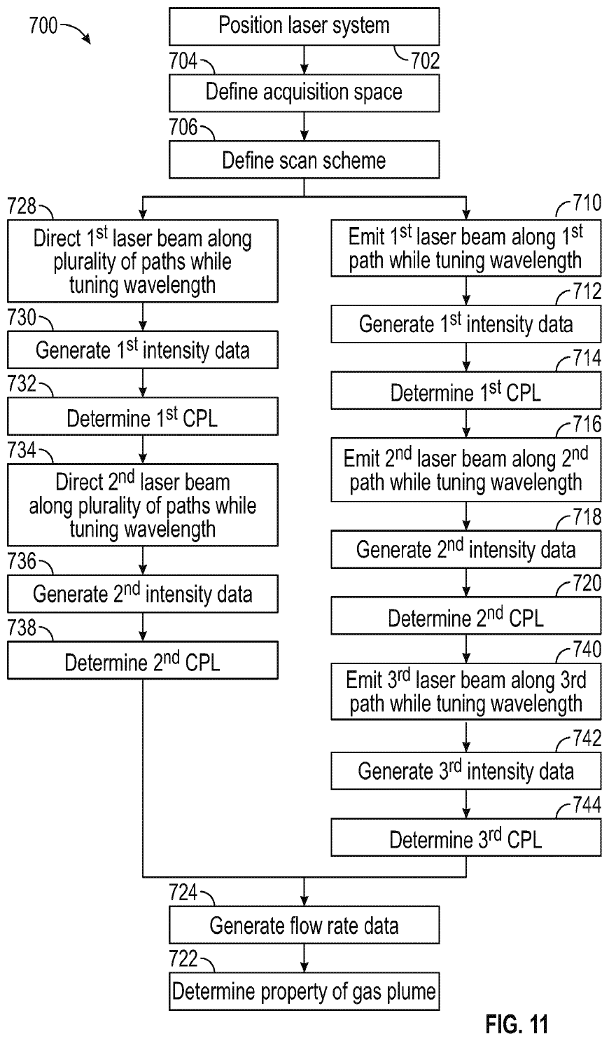

FIG. 11 is a flow-chart diagram of at least a portion of an example implementation of a method (700) for determining a property of a gas plume 302 produced by burning of a hydrocarbon effluent via a burning device 306. The method (700) may be performed or otherwise implemented via or otherwise in conjunction with at least a portion of one or more implementations of one or more instances of the apparatus shown in one or more of FIGS. 1-10. Accordingly, the following description refers to FIGS. 1-11, collectively.

The method (700) may comprise positioning (702) a laser system 320 in association with the burning device 306 or at another location such that the gas plume 302 is in a field of view of the laser system 302. The method (700) may further include defining (704) an acquisition space 303 (e.g., an air column) comprising at least a portion of the gas plume 302 through which the laser system 320 may emit laser beams 330 to analyze the gas plume 302. The method (700) may further comprise defining (706) a scheme for scanning the space 303 with the laser beams 330.

The method (700) may further comprise emitting (710) a first laser beam 330 along a first path 305 through the acquisition space 303 containing at least a portion of the gas plume 302 while tuning the wavelength of the first laser beam 330 around a first wavelength 408 corresponding to a spectral absorption line of a first predetermined gas, generating (712) first intensity data (or signal) 402 indicative of intensity 404 of backscatter 332 of the first laser beam 330, and determining (714) a first concentration path length (CPL) of the first predetermined gas along the first path 305 based on the first intensity data. The method (700) may further comprise emitting (716) a second laser beam 330 along a second path 332 through the gas plume 302 while tuning the wavelength of the second laser beam 330 around a second wavelength 408 corresponding to a spectral absorption line of a second predetermined gas, generating (718) second intensity data (or signal) 402 indicative of intensity 404 of backscatter 332 of the second laser beam 330, and determining (720) a second concentration path length (CPL) of the second predetermined gas along the second path 305 based on the second intensity data. The first predetermined gas may be CO₂ and the second predetermined gas may be CO, CO₂, CH₄, C₂H₂, C₂H₄, C₃H₆, NO, NO₂, NO₃, or SO₂.

The method (700) may further comprise determining (722) the property of the gas plume 302 based on a relationship between the first and second concentration path lengths. The relationship between the first and second concentration path lengths may comprise a ratio of the second concentration path length to the first concentration path length.

The method (700) may comprise directing (728) the first laser beam along a plurality of first paths through the acquisition space 303 containing at least a portion of the gas plume 302 and, for each of the first paths through the gas plume, tuning the wavelength of the first laser beam 330 around the first wavelength 408 corresponding to the spectral absorption line of the first predetermined gas, generating (730) the first intensity data indicative of the backscatter intensity, and determining (732) the first concentration path length of the first predetermined gas based on the first intensity data. The method may further comprise directing (734) the second laser beam along a plurality of second paths through the acquisition space 303 containing the at least a portion of the gas plume 302 and, for each of the second paths through the gas plume 302, tuning the wavelength of the second laser beam 330 around the second wavelength 408 corresponding to the spectral absorption line of the second predetermined gas, generating (736) the second intensity data indicative of the backscatter intensity, and determining (738) the second concentration path length of the second predetermined gas based on the second intensity data. Determining (722) the property of the gas plume may be based on a relationship between the first concentration path lengths and the second concentration path lengths. The relationship between the first concentration path lengths and the second concentration path lengths may comprise a relationship between an average of the first concentration path lengths and an average of the second concentration path lengths.

The property of the gas plume 302 may be or comprise a rate of emission of the second predetermined gas during the burning of the hydrocarbon effluent. The method (700) may further comprise generating (724) flow rate data indicative of a volumetric and/or a mass flow rate of the hydrocarbon effluent flowing to the burning device 306. When determining the rate of emission of the second predetermined gas, determining (722) the property of the gas plume may be based further on the flow rate data. Determining (722) the property of the gas plume may be based further on the flow rate data, the density of the hydrocarbon effluent, and the molar mass of the hydrocarbon effluent.

The property of the gas plume 302 may be or comprise combustion efficiency of the hydrocarbon effluent. When the second predetermined gas has a composition comprising carbon, the method (700) may comprise determining (722) the property of the gas plume based on a ratio of the first concentration path length to a sum of the first and second concentration path lengths. When the second predetermined target gas is CH₄, the method (700) may also or instead comprise emitting (740) a third laser beam 330 along a third path 305 through the gas plume 302 while tuning the wavelength of the third laser beam 330 around a third wavelength 408 corresponding to a spectral absorption line of a third predetermined gas. The third predetermined gas may be CO. The method (700) may further comprise generating (742) third intensity data (or signal) 402 indicative of intensity 404 of backscatter 332 of the third laser beam 330, and determining (744) a third concentration path length of the third predetermined gas along the third path 305 based on the third intensity data. When determining combustion efficiency, determining (722) the property of the gas plume may be based on a ratio of the first concentration path length to a sum of the first, second, and third concentration path lengths.

A gas monitoring system (e.g., the gas monitoring system 300, 500) according to one or more aspects of the present disclosure may be further operable to accurately determine (e.g., calculate, estimate, etc.) various properties of a gas plume (e.g., a mean concentration path length of each component gas, combustion efficiency of the hydrocarbon effluent during burning operations, etc.). To perform such operations, the gas monitoring system may be operable to perform qualitative analysis of a concentration path length map of a scanned space that encompasses (or extends through) at least a portion of the gas plume. Such qualitative analysis may include determining location of the plume on the concentration path length map by defining a plume region (i.e., a bit mask or area) of the concentration path length map comprising pixels covering or otherwise associated with the gas plume, and then determining the properties of the gas plume based on concentration path length data (i.e., concentration path length measurements) within such plume region. Accordingly, the present disclosure is further directed to a method of processing concentration path length data to find or otherwise determine the plume region on the concentration path length map and to use the concentration path length data within such plume region to determine properties of one or more gases within the plume. Such processing method may be implemented via a computer algorithm that can be automatically executed by a processing device of a gas monitoring system according to one or more aspects of the present disclosure.

An algorithm according to one or more aspects of the present disclosure may cause a processing device (e.g., the processing device 336, 352, 364, 600) to receive concentration path length data from a laser system (e.g., the laser system 320), and determine (or output) mean (or average) concentration path lengths of component gases (e.g., methane, carbon dioxide, etc.) in a combustion plume and/or a combustion efficiency of a hydrocarbon effluent. The processing device may receive the concentration path length data that has been discretized in the form of pixels of a concentration path length map. The algorithm may cause the processing device to determine (or estimate) a plume region (i.e., a bit mask or area) of pixels of the concentration path length map by identifying a background region (i.e., a bit mask or area) of pixels that do not include the plume region (i.e., do not cover or otherwise comprise the gas plume) of the concentration path length map. The algorithm may further cause the processing device to apply a statistical test to sample batches of concentration path length data to determine if such data is part of the background region or a part of the plume region of the concentration path length map. Based on such analysis, the processing device may be operable to determine the plume region of the concentration path length map and refine estimates (or calculations) of the background region, while at the same time using the determined plume region to determine mean concentration path lengths of subsequent sample batches of concentration path length data. It is to be noted that the algorithm may be first used on concentration path length data indicative of carbon dioxide, as carbon dioxide is the main product of effluent combustion and is always present in large quantity in a combustion gas plume. The determined plume region may then be used with subsequent sample batches of concentration path length data for both methane and carbon dioxide to determine the mean concentration path length of each gas and then compute the combustion efficiency.

An algorithm according to one or more aspects of the presented disclosure may comprise or otherwise use a plurality of independent parameters to facilitate determination of a property of a gas plume. One (e.g., a first) of the parameters (referred to hereinafter as parameter delta-time-background (DT_B)) may define a processing time window (or duration) for determining a mean background concentration path length of a concentration path length map based on concentration path length data within the background region of the concentration path length map. The value of the parameter DT_B may range, for example, between about 2.0 minutes and 10.0 minutes, between about 4.0 minutes and 6.0 minutes, between about 4.5 minutes and 5.5 minutes, or a longer period of time. The value of the parameter DT_B may be, for example, 3.0 minutes, 4.0 minutes, 5.0 minutes, 6.0 minutes, or longer. Another (e.g., a second) of the parameters (referred to hereinafter as parameter delta-time-concentration (DT_C)) may define a processing time window (or duration) for determining a mean plume concentration path length of the concentration path length map based on concentration path length data within the plume region of the concentration path length map. The value of the parameter DT_C may range, for example, between about 1.0 minute and 3.0 minutes, between about 1.5 minutes and 2.5 minutes, between about 2.0 minutes and 2.5 minutes, or a longer period of time. The value of the parameter DT_C may be, for example, 1.0 minute, 1.5 minutes, 2.0 minutes, 2.5 minutes, or longer. Another (e.g., a third) of the parameters (referred to hereinafter as parameter delta-time-sampling (DT_S)) may define a sampling time window (or duration) between processing successive time windows DT_B and DT_C. The value of the parameter DT_S may range, for example, between about 1.0 minute and 3.0 minutes, between about 1.5 minutes and 2.5 minutes, or between about 2.0 minutes and 2.5 minutes. The value of the parameter DT_S may be, for example, 1.0 minute, 1.5 minutes, 2.0 minutes, or 2.5 minutes. However, the value of the parameter DT_S may be decreased or increased depending on data and intended output sampling rate.

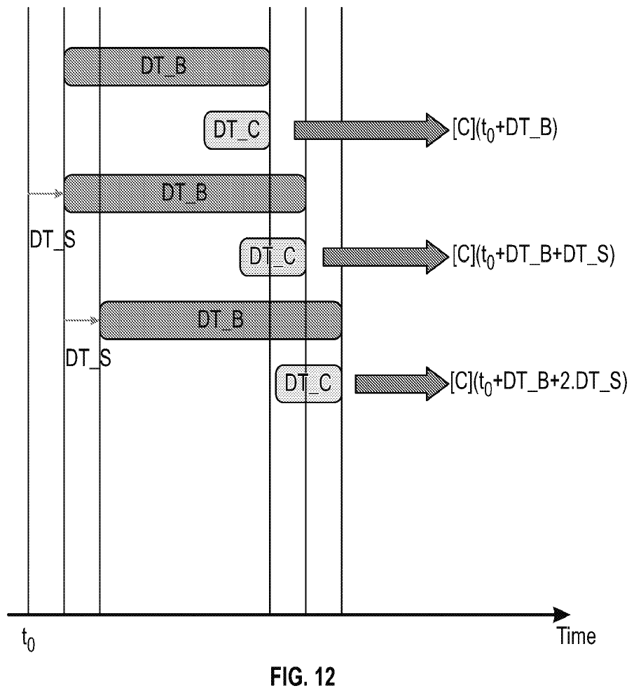

FIG. 12 is a schematic view (or visual representation) of the three-time window parameters, DT_B, DT_C, and DT_S with respect to time, shown along a horizontal axis. Each successive plurality of operations to determine a property of the gas plume (performed during corresponding processing time windows DT_B and DT_C) may be performed every sampling time window DT_S, starting at time to. As shown in FIG. 12, if DT_S <DT_C, then concentration path length data points can be reused in multiple processing time windows DT_C. However, if DT_S>=DT_C, then there is no overlap between the processing time windows DT_C.

Another (e.g., a fourth) of the parameters (referred to hereinafter as parameter sigma-threshold (K_{threshold})) may define a sigma value indicative of predetermined quantity of standard deviations of a distribution of the concentration path length data points with respect to a mean concentration path length. The parameter K_{threshold} may be used in statistical test for the classification of concentration path length data points, wherein some concentration path length data points may be associated (or aligned) with the background region, and some of the concentration path length data points may be associated (or aligned) with the plume region. The value of the parameter K_{threshold} may range, for example, between about 1.0 and 4.00, between about 1.25 and 3.0, or between about 1.5 and 2.0. The value of the parameter K_{threshold} may be, for example, 1.5, 1.75, or 2.0.

Still another (e.g., a fifth) of the parameters (referred to hereinafter as parameter number-grid (N_{grid})) may define size (e.g., height and/or width) of the concentration path length map comprising the discretized concentration path length data. For example, the parameter N_{grid} may be or comprise the quantity of vertical and/or horizontal pixels forming the concentration path length map. The value of the parameter N_{grid} may range, for example, between about 10 and 40, between about 15 and 30, or between about 20 and 25. The value of the parameter N_{grid} may be, for example, 15, 20, 25, 30, 35, or 40. However, the value of the parameter N_{grid} may be higher.

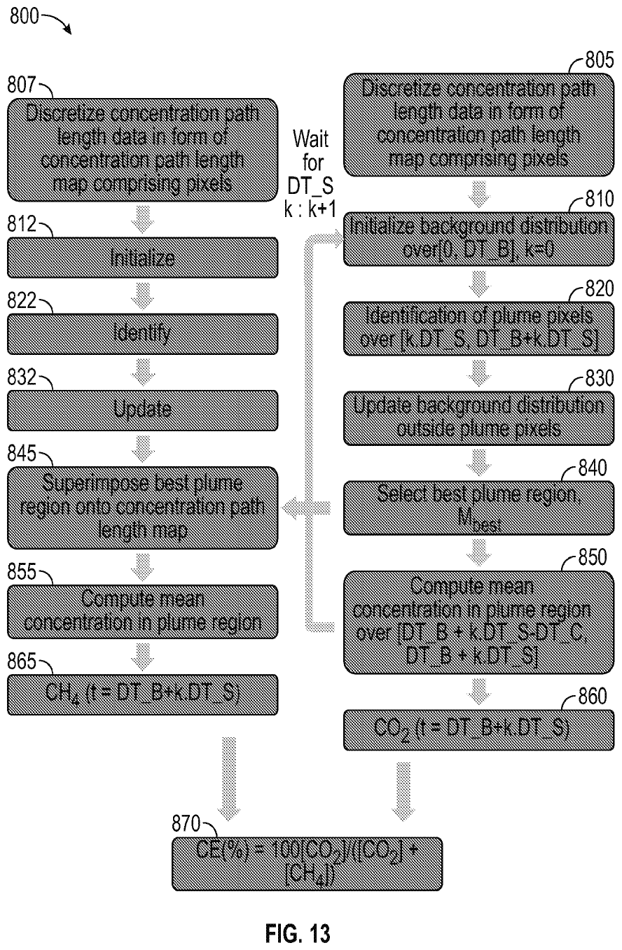

FIG. 13 is a flow-chart diagram of at least a portion of an algorithm 800 for performing or otherwise facilitating a workflow according to one or more aspects of the present disclosure. The algorithm 800 may be performed or otherwise implemented via or otherwise in conjunction with at least a portion of one or more implementations of one or more instances of the apparatus shown in one or more of FIGS. 1-10. For example, the algorithm 800 may be caused to be performed by one or more processing devices (e.g., the processing devices 336, 352, 364, 600) receiving concentration path length data generated by a laser system (e.g., the laser system 320) according to one or more aspects of the present disclosure.

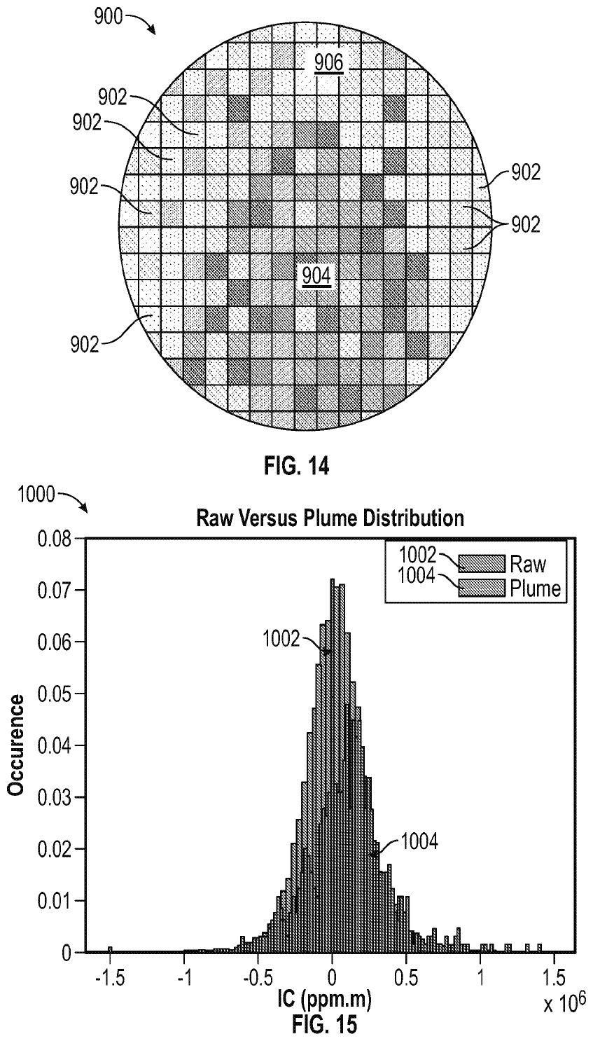

The algorithm 800 may comprise discretizing 805 concentration path length data output by a laser system (e.g., the laser system 320 shown in FIG. 4) in the form of discretized pixels collectively forming a concentration path length map. FIG. 14 shows an example concentration path length map 900 (hereinafter “concentration map”) comprising a plurality of vertical columns and horizontal rows of discretized pixels 902 indicative of or otherwise based on the concentration path lengths of a gas within a defined space scanned by a laser system. Each pixel 902 may be indicative of or otherwise based on one or more concentration path length data points, and each concentration path length data point may be indicative of a determined concentration path length measurement of the component gas within the defined space scanned by the laser system. The concentration map 900 may be generated by one or more processing devices described herein.

The algorithm 800 may further comprise initializing (or estimating) 810 a distribution of background concentration path length data of a concentration path length map over a time window [0, DT_B], which includes receiving (i.e., gathering) and processing concentration path length data over the first DT_B minutes of the workflow between time zero (0) and time DT_B. The processing of the concentration path length data may include fitting a Gaussian mixture model (GMM) with two components, such as a mean concentration path length μ_{background} of the background region (hereinafter “mean background concentration”) and a standard deviation of distribution σ_{background} of the concentration path length data points of the background region (hereinafter “background standard deviation”). The processing of the concentration path length data may further include selecting the Gaussian mixture model with the lowest mean background concentration μ_{background} as the mean background concentration μ_{background}. The initializing 810 of the background concentration path length distribution may comprise processing of the concentration path length data of every pixel 902 forming the concentration path length map 900, such that the mean background concentration μ_{background} and the background standard deviation σ_{background} is based on the concentration path length data of every pixel 902 forming the concentration path length map.

Although the Gaussian mixture model can be used, it is to be noted that the mean background concentration μ_{background} and the mean background standard deviation σ_{background} may be assessed using other statistical methods. Furthermore, the selection of the mean background concentration μ_{background} and the mean background standard deviation σ_{background} using the GMM may be improved, for example, by merging two data points, when values of such two data points are very close. If needed, the selection of the mean background concentration μ_{background} and the mean background standard deviation σ_{background} may also be performed later in the algorithm 800, such as at a background estimation stage. Also, because certain portions of the algorithm 800 may be repeated, the initializing 810 may further include setting a repetition counter k to zero (0).

The algorithm 800 may further comprise identifying 820 pixels 902 of the

concentration map 900 associated with the gas plume over the time window [k.DT_S, DT_B+k.DT_S]. The identifying 820 operation may comprise receiving and processing by a processing device concentration path length data between time k.DT_S and time DT_B+k.DT_S of the workflow. As described above, each concentration path length data point may be represented by or associated with a pixel 902 of the concentration map 900. The processing of the concentration path length data may include splitting the pixels 902 of the concentration map 900 based on their location on the concentration map 900. Each pixel 902 may comprise a quantity N_{observation} (i,j) of concentration path length data points (or observations), which form or comprise a concentration path length data sample having a mean concentration path length μ_{sampling} (i,j) (hereinafter “mean concentration”). The processing of the concentration path length data points may include computing a standard deviation σ_{sampling} (i,j) of a distribution of the concentration path length data points of each pixel 902 using a standard deviation formula as set forth in Equation (19):

$${σ}_{sampling} ( i , j ) = \frac{{σ}_{background}}{\sqrt{{N}_{observation} ( i , j )}} \qquad ( 19 )$$

The processing of the concentration path length data may further include classifying each pixel 902 as a plume pixel (i.e., a pixel 902 that is a part of or forms a plume region 904 of the concentration map 900), if the mean concentration path length μ_{sampling} (i,j) is grater or equal to the product of the sigma-threshold K_{threshold} and the standard deviation σ_{sampling} (i,j) as set forth in Equation (20):

$${μ}_{sampling} ( i , j ) \geq {K}_{threshold} * {σ}_{sampling} ( i , j ) \qquad ( 20 )$$

Each pixel 902 that is not classified as a plume pixel may be classified as a background pixel (i.e., a pixel 902 that is not a part of the plume region 904 and, thus, is a part of or forms a background region 906 of the concentration map 900).

The algorithm 800 may further comprise updating (or recalculating) 830 the distribution of the background pixels (including updating the mean background concentration μ_{background} and the mean background standard deviation σ_{background}) that have been previously initialized 810. The updating 830 operation may comprise determining the mean background concentration μ_{background} and the mean background standard deviation σ_{background} in the same or similar manner as described above with respect to the initializing 810 operation, except that the updating 830 operation considers or is otherwise based just on the background pixels of the background region 906 identified during the identification 820 operation. In other words, the updating 830 operation may comprise recalculating the mean background concentration μ_{background} and the mean background standard deviation σ_{background} using or otherwise based just on the background pixels of the background region 906 (i.e., pixels 902 that are outside of the plume region 904). Because the mean background concentration μ_{background} is indicative of concentration path lengths in the background region 906, which does not include the combustion plume comprising the predetermined gas, the mean background concentration σ_{background} may thus be or comprise background noise in the concentration path length measurements and not actual concentration path length measurements of the predetermined gas.

The algorithm 800 may further comprise selecting 840 a best plume region M_{best}. The selecting 840 operation may include selecting pixels (or pixel groups) 902 that are connected (or adjacent) to the plume pixels of the plume region 904 and designating such pixels 902 as plume pixels. The selecting 840 operation may use a simple score to select a connected pixel group with the largest number of high concentration path length data points. For example, the selecting 840 operation may further include ranking each connected pixel group using a selection formula as set forth in Equation (21):

$$S = {\sum }_{i} {N}_{i} ( {μ}_{i} - {μ}_{background} ) \qquad ( 21 )$$

where Nᵢ is a quantity of concentration path length data points (or observations) in or otherwise associated with a pixel i and μᵢ is a mean concentration path length of or otherwise associated with the pixel i. Each plume pixel selection value S′ may thus be determined based on the difference between the mean concentration path length μᵢ of the plume pixel and the mean background noise (i.e., the mean background concentration μ_{background}). The selecting 840 operation may further include selecting the pixels (or pixel groups) 902 with the largest score to determine the best plume region M_{best}. The pixels 902 of the best plume region M_{best} may be referred to as a plume mask.

FIG. 15 is a graph 1000 showing distribution of the concentration path length data points 1002 of the pixels 902 of the entire concentration map 900 (i.e., of the plume region 904 and the background region 906) and concentration path length data points 1004 of the pixels 902 of just the plume region 904. The horizontal axis shows the concentration path length of the concentration path length data points in units of PPM*M, and the vertical axis shows proportion (or fraction) of occurrence of each concentration path length value. The concentration path length data points 1002 are shown normalized with respect to 0 PPM*M. The concentration path length data points 1004 are shown centered on the positive side of 0 PPM*M.

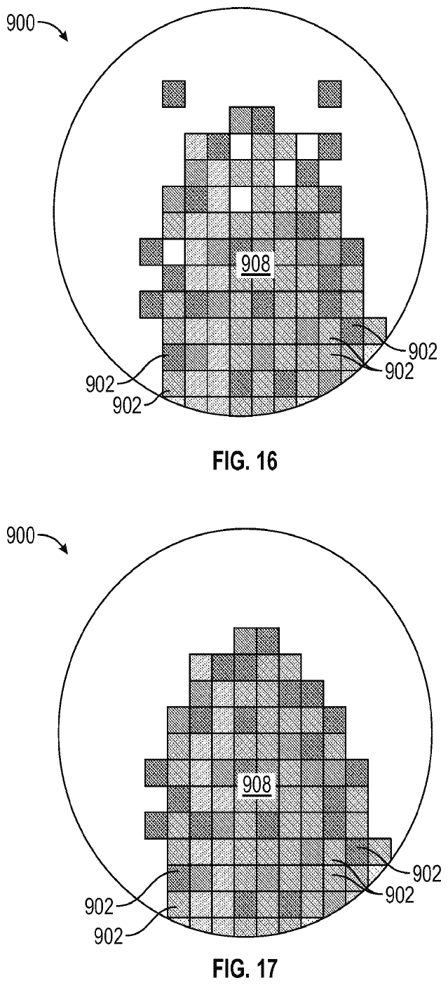

FIG. 16 shows just the selected pixels of the best plume region M_{best} 908, wherein the pixels of the background region 906 are discarded, filtered out, or otherwise not considered. A data filter (e.g., a median filter) may be applied to the best plume region M_{best} to fill gaps between the pixels of the best plume region M_{best} and/or to discard disconnected pixels of the best plume region M_{best}. FIG. 17 shows the pixels of the best plume region M_{best} 908, wherein gaps between the pixels of the best plume region M_{best} have been filled and disconnected pixels of the best plume region M_{best} have been discarded by the data filter.

The algorithm 800 may further comprise determining (or computing) 850 the mean concentration path length μ_{sampling} (i,j) of the concentration path length data associated with pixels 902 of the best plume region M_{best} 908 over the time window [DT_B+k.DT_S-DT_C, DT_B+k.DT_S]. The determining 850 operation may comprise receiving (i.e., gathering) and processing concentration path length data between time DT_B+k.DT_C-DT_C and time DT_B+k.DT_S of the workflow. The processing of the concentration path length data may comprise segregating (or splitting) the concentration path length data into pixels 902 based on their position on the concentration map 900, selecting the concentration path length data contained in the plume pixels of the best plume region M_{best} 908, and determining the mean concentration path length μ_{sampling} (i,j) and the standard deviation σ_{sampling} (i,j) of distribution of such concentration path length data.

The algorithm 800 may also comprise outputting 860 the calculated (or recalculated) 850 mean concentration path length μ_{sampling} (i,j) for further analysis. The operations 820, 830, 840, 850 may be repeated every time window DT_S and a predetermined k number of times, each time updating (i.e., recalculating) the mean concentration path length μ_{sampling} (i,j) and standard deviation σ_{sampling} (i,j) of the best plume region M_{best} 908 and increasing the repetition counter k by a value of one (1). Each time the operations 820, 830, 804, 850 of the algorithm 800 are repeated, a new (i.e., updated or recalculated) frame of the concentration map 900 comprising pixels 902 having new mean concentration path length μ_{sampling} (i,j) and standard deviation σ_{sampling} (i,j) values.

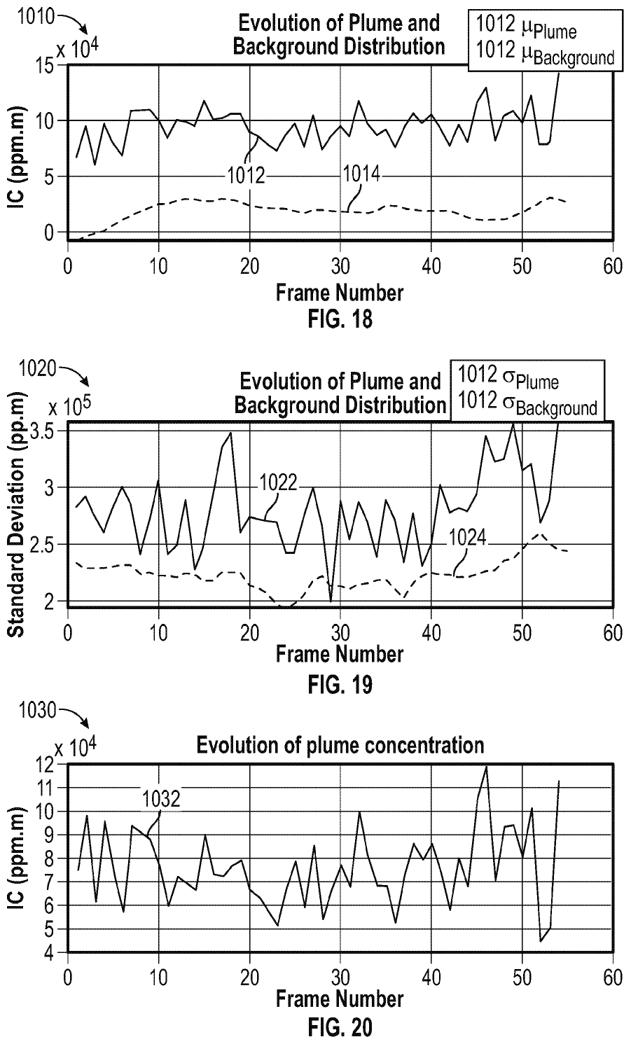

FIG. 18 is a graph 1010 showing evolution of the mean concentration μ_{sampling} (i,j) 1012 of the concentration path length data of the plume pixels of the best plume region M_{best} 908 and the mean concentration μ_{sampling} (i,j) 1014 of the concentration path length data of the background pixels of the background region 906. The mean concentrations μ_{sampling} (i,j) 1012, 1014, shown along the vertical axis of the graph 1010, are depicted with respect to each successive frame of the concentration map 900, shown along the horizontal axis of the graph 1010.

FIG. 19 is a graph 1020 showing evolution of the standard deviation σ_{sampling} (i,j) 1022 of the concentration path length data of the plume pixels of the best plume region M_{best} 908 and the standard deviation σ_{sampling} (i,j) 1024 of the concentration path length data of the background pixels of the background region 906. The standard deviations σ_{sampling} (i,j) 1022, 1024, shown along the vertical axis of the graph 1020, are depicted with respect to each successive frame of the concentration map 900, shown along the horizontal axis of the graph 1020.

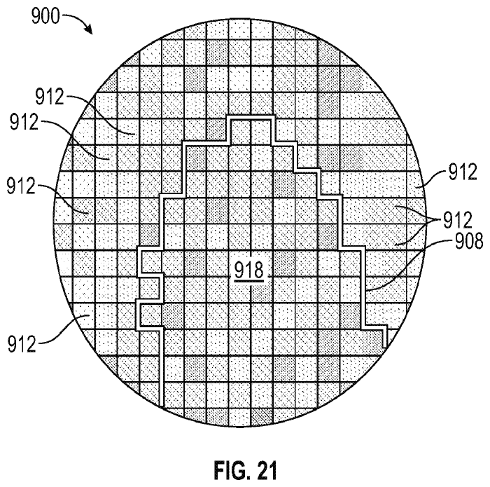

FIG. 20 is a graph 1030 showing evolution of the mean concentration μ_{sampling} (i,j) 1032 of the concentration path length data of the plume pixels of the best plume region M_{best} 908 above (or with respect to) the mean concentration μ_{sampling} (i,j) 1014 (shown in FIG. 18) of the concentration path length data of the background pixels of the background region 906. In other words, the mean concentration μ_{sampling} (i,j) 1032 is the difference between the mean concentration μ_{sampling} (i,j) 1012 and the mean concentration μ_{sampling} (i,j) 1014 (i.e., the background noise). The mean concentration μ_{sampling} (i,j) 1032, shown along the vertical axis of the graph 1030, is depicted with respect to each successive frame of the concentration map 900, shown along the horizontal axis of the graph 1030.

It is to be noted, that the workflow facilitated by the algorithm 800 may be first used with respect to just carbon dioxide gas (i.e., carbon dioxide concentration path length data) to determine location of a combustion gas plume (i.e., determine the best plume region M_{best} 908) as carbon dioxide is the main product of effluent combustion and is always present in large quantity in the plume while determining the mean concentration path length of carbon dioxide. Then, after the best carbon dioxide plume region M_{best} 908 and the mean concentration μ_{sampling} (i,j) of carbon dioxide is determined, the algorithm 800 may be used with respect to another predetermined gas (e.g., methane) to receive concentration path length data associated with the other gas and then determine the mean concentration μ_{sampling} (i,j) of the other gas.

The algorithm 800 may thus further comprise discretizing 807 concentration path length data associated with another predetermined gas output by a laser system (e.g., the laser system 320 shown in FIG. 4) in the form of discretized pixels to form a concentration path length map. FIG. 21 shows an example concentration path length map 910 (hereinafter “concentration map”) comprising a plurality of vertical columns and horizontal rows of discretized pixels 912 indicative of or otherwise based on the concentration path length data of methane gas within the defined space scanned by the laser system. Each pixel 912 may be indicative of or otherwise based on one or more concentration path length data points, and each concentration path length data point may be indicative of a determined concentration path length measurement of methane within the defined space scanned by the laser system.

The algorithm 800 may further comprise initializing 812 a distribution of background concentration path length data of the concentration path length map 910, identifying 822 pixels 912 of the concentration map 910, and updating 832 the distribution of the background pixels that have been previously initialized 812. The initializing 812, the identifying 822, and the updating 832 operations for the methane gas may be implemented in the same or similar manner as the initializing 810, the identifying 820, and the updating 830 operations, respectively, described above.

The algorithm 800 may further comprise processing the concentration path length data of the concentration map 910 of methane to compute (or determine) 855 the mean μ_{sampling} (i,j) and standard deviation σ_{sampling} (i,j) of the methane concentration path length data within the pixels 912 of a best methane plume region M_{best} 918. The best methane plume region M_{best} 918 may be determined (or selected) by superimposing or otherwise applying 845 the best carbon dioxide plume region M_{best} 908 of carbon dioxide to the concentration map 910 of methane and designating the pixels 912 within or encompassed by the best carbon dioxide plume region M_{best} 908 as the pixels 912 of the best methane plume region M_{best} 918. The processing device may then compute 855 the mean concentration μ_{sampling} (i,j) and standard deviation σ_{sampling} (i,j) of the methane concentration path length data within the pixels 912 of the best methane plume region M_{best} 918 in the same or similar manner as described above with respect to operation 850. The best methane plume region M_{best} 918 and the mean μ_{sampling} (i,j) of the methane concentration path length data may be recalculated 855 every time the best carbon dioxide plume region M_{best} 908 recalculated 840. The algorithm 800 may also comprise outputting 865 the calculated (or recalculated) 855 mean concentration path length μ_{sampling} (i,j) of methane for further analysis.

It is to be noted that the algorithm 800 may be applied to carbon dioxide and methane components of a gas plume at different times, as described above. However, it is to be further noted that the algorithm 800 may instead be applied simultaneously with respect to both carbon dioxide and methane of the gas plume to determine the mean μ_{sampling} (i,j) concentration path lengths of carbon dioxide and methane. It is to be still further noted that the algorithm 800 may be used on combined data comprising both carbon dioxide gas and methane gas concentration path length data to simultaneously determine the mean μ_{sampling} (i,j) concentration path length of carbon dioxide and methane.

In an example implementation, the mean concentrations μ_{sampling} (i,j) of carbon dioxide and methane may be output to or received by a processing device, such as to determine 870 efficiency of combustion of the hydrocarbon effluent. For example, the mean μ_{sampling} (i,j) concentration path length of carbon dioxide and the mean μ_{sampling} (i,j) concentration path length of methane may be received by the processing device, which may then use such data and Equation (18) to determine efficiency of combustion of a hydrocarbon effluent. If the mean μ_{sampling} (i,j) of the methane concentration path length data of the pixels 912 within the best methane plume region M_{best} 918 is indicative of low or no presence of methane, then the combustion of the hydrocarbon effluent may be considered as complete or otherwise highly efficient.

The foregoing outlines features of several embodiments so that a person having ordinary skill in the art may better understand the aspects of the present disclosure. A person having ordinary skill in the art should appreciate that they may readily use the present disclosure as a basis for designing or modifying other processes and structures for carrying out the same functions and/or achieving the same benefits of the embodiments introduced herein. A person having ordinary skill in the art should also realize that such equivalent constructions do not depart from the spirit and scope of the present disclosure, and that they may make various changes, substitutions and alterations herein without departing from the spirit and scope of the present disclosure.

1. A gas monitoring system for determining a property of a gas plume produced by burning of a hydrocarbon effluent via a burning device, wherein the gas monitoring system comprises:
a laser emission system operable to emit a laser beam along a plurality of paths passing through the gas plume;
a detection system operable to facilitate determining intensity data indicative of intensities of the laser beam that has been backscattered by a surface after passing through the gas plume; and
a processing system comprising a processor and a memory device storing a computer program code that, when executed by the processor, causes the processing system to:
cause the laser emission system to emit the laser beam along the path;
output concentration path length data indicative of concentration path lengths of a predetermined gas along the paths based on the intensity data;
discretize the concentration path length data in the form of a concentration path length map, wherein the concentration path length map comprises a plurality of pixels representing the concentration path length data;
find a plume region of the concentration path length map comprising instances of the pixels associated with the gas plume; and
determine mean concentration path length of the predetermined gas based on the concentration path length data of the pixels of the plume region.

2. The gas monitoring system of claim 1 wherein, the computer program code, when executed by the processor, further causes the processing system to:
determine a mean concentration path length based on the concentration path length data of the pixels; and
determine a mean concentration standard deviation of the concentration path length of each instance of the pixels.

3. The gas monitoring system of claim 1 wherein, to find the plume region of the concentration path length map comprising instances of the pixels associated with the gas plume, the processing system is operable to, for each instance of the pixels:
determine a pixel concentration standard deviation of a distribution of the concentration path lengths of the instance of the pixel;
determine a mean pixel concentration path length of the concentration path lengths of the instance of the pixel;
compare the mean pixel concentration path length to a product of the pixel concentration standard deviation and a predetermined threshold standard deviation; and
classify the instance of the pixel as being within the plume region when the mean pixel concentration path length is greater than the product of the pixel concentration standard deviation and the predetermined threshold standard deviation.

4. The gas monitoring system of claim 1 wherein, the computer program code, when executed by the processor, further causes the processing system to:
find a background region of the concentration path length map comprising instances of the pixels not associated with the gas plume; and
determine mean concentration path length of the predetermined gas based on the concentration path length data of the pixels of the background region.

5. The gas monitoring system of claim 1 wherein:
the predetermined gas is a first predetermined gas;
the concentration path length data is a first concentration path length data;
the concentration path length map is a first concentration path length map;
the plurality of pixels is a plurality of first pixels;
the mean concentration path length is a first mean concentration path length; and
the computer program code, when executed by the processor, further causes the processing system to:
determine second concentration path length data indicative of the concentration path length of a second predetermined gas along the path based on the intensity data;
discretize the second concentration path length data in the form of a second concentration path length map, wherein the second concentration path length map comprises a plurality of second pixels representing the second concentration path length data;
superimpose the plume region of the first concentration path length map onto the second concentration path length map to encompass instances of the second pixels; and
determine second mean concentration path length of the second predetermined gas based on the second concentration path length data of the second pixels within the plume region.

6. The gas monitoring system of claim 5 wherein the first predetermined gas in carbon dioxide (CO₂), and wherein the second predetermined gas is methane (CH₄).

7. The gas monitoring system of claim 5 wherein, the computer program code, when executed by the processor, further causes the processing system to determine efficiency of the burning to the hydrocarbon effluent based on the first mean concentration path length of the first predetermined gas and the second mean concentration path length of the second predetermined gas.

8. A gas monitoring system for determining a property of a gas plume produced by burning of a hydrocarbon effluent via a burning device, wherein the gas monitoring system comprises:
a laser emission system operable to emit first and second laser beams along a path passing through the gas plume;
a detection system operable to facilitate determining first and second intensity data indicative of intensities of the first and second laser beams, respectively, that have been backscattered by a surface after passing through the gas plume; and
a processing system comprising a processor and a memory device storing a computer program code that, when executed by the processor, causes the processing system to:
cause the laser emission system to emit the first laser beam along the path while tuning wavelength of the first laser beam around a first wavelength corresponding to a spectral absorption line of a first predetermined gas, wherein the first predetermined gas is carbon dioxide (CO₂);
cause the laser emission system to emit the second laser beam along the path while tuning wavelength of the second laser beam around a second wavelength corresponding to a spectral absorption line of a second predetermined gas;
determine first concentration path lengths of the first predetermined gas along the paths based on the first intensity data;
output first concentration path length data indicative of the first concentration path lengths;
discretize the first concentration path length data in the form of a first concentration path length map, wherein the first concentration path length map comprises a plurality of first pixels representing the first concentration path length data;
find a plume region of the first concentration path length map comprising instances of the first pixels associated with the gas plume;
determine mean first concentration path length of the first predetermined gas based on the first concentration path length data of the first pixels of the plume region;
determine second concentration path lengths of the second predetermined gas along the paths based on the second intensity data;
output second concentration path length data indicative of the second concentration path lengths;
discretize the second concentration path length data in the form of a second concentration path length map, wherein the second concentration path length map comprises a plurality of second pixels representing the second concentration path length data;
superimpose the plume region of the first concentration path length map onto the second concentration path length map to encompass instances of the second pixels; and
determine second mean concentration path length of the second predetermined gas based on the second concentration path length data of the second pixels within the plume region.

9. The gas monitoring system of claim 8 wherein, the computer program code, when executed by the processor, further causes the processing system to:
determine a mean concentration path length of the first predetermined gas based on the first concentration path length data of the first pixels; and
determine a mean concentration standard deviation of the first concentration path lengths of the first pixels.

10. The gas monitoring system of claim 8 wherein, to find the plume region of the first concentration path length map comprising instances of the first pixels associated with the gas plume, the processing system is operable to, for each instance of the first pixels:
determine a pixel concentration standard deviation of a distribution of the concentration path lengths of the instance of the first pixel;
determine a mean pixel concentration path length of the concentration path lengths of the instance of the first pixel;
compare the mean pixel concentration path length to a product of the pixel concentration standard deviation and a predetermined threshold standard deviation; and
classify the instance of the first pixel as being within the plume region when the mean pixel concentration path length is greater than the product of the pixel concentration standard deviation and the predetermined threshold standard deviation.

11. The gas monitoring system of claim 8 wherein, the computer program code, when executed by the processor, further causes the processing system to:
find a background region of the first concentration path length map comprising instances of the first pixels not associated with the gas plume; and
determine mean background concentration path length of the predetermined gas based on the first concentration path length data of the first pixels of the background region.

12. The gas monitoring system of claim 8 wherein the second predetermined gas is methane (CH₄).

13. The gas monitoring system of claim 8 wherein, the computer program code, when executed by the processor, further causes the processing system to determine efficiency of the burning to the hydrocarbon effluent based on the first mean concentration path length of the first predetermined gas and the second mean concentration path length of the second predetermined gas.

14-18. (canceled)
